(19) United States
(12) Patent Application Publication
(10) Pub. No.: US 2018/0055372 A1
(43) Pub. Date: Mar. 1, 2018

(54) IMAGE PROCESSING APPARATUS AND ENDOSCOPE APPARATUS

(71) Applicant: OLYMPUS CORPORATION, Tokyo (JP)

(72) Inventors: Takeshi WATANABE, Tokyo (JP); Makoto IGARASHI, Tokyo (JP); Mitsuru NAMIKI, Saitama (JP); Hiroyoshi YAJIMA, Kanagawa (JP); Yuji HIRAI, Kanagawa (JP); Satoshi HONDA, Tokyo (JP); Kazuhiro TANAKA, Tokyo (JP); Yusuke TAKEI, Tokyo (JP)

(73) Assignee: OLYMPUS CORPORATION, Tokyo (JP)

(21) Appl. No.: 15/789,004

(22) Filed: Oct. 20, 2017

Related U.S. Application Data
(63) Continuation of application No. PCT/JP2016/062830, filed on Apr. 22, 2016.
(60) Provisional application No. 62/151,585, filed on Apr. 23, 2015.

(51) Int. Cl. A61B 5/02; A61B 1/04; A61B 1/00; A61B 5/00; G06T 7/00; G06T 7/70; H04N 5/225

(52) U.S. Cl. CPC A61B 5/02; A61B 1/04; A61B 1/00045; A61B 5/7278; H04N 2005/2255; G06T 7/70; H04N 5/2256; G06T 2207/30101; G06T 2207/30204; G06T 7/0012

(57) ABSTRACT

An image processing apparatus including: an image acquisition device for observing a living body; a blood vessel recognition device that irradiates the living body with an exploration laser beam and that recognizes a blood vessel; an arithmetic operation unit that calculates a first laser spot position on a first frame acquired by the image acquisition device when the blood vessel is recognized and that adds a mark corresponding to the first laser spot to a position on a second frame corresponding to the first frame, the second frame being acquired at a time different from the time of the first frame; and a display unit for displaying, on an image acquired by the image acquisition device, a plurality of the marks added by the arithmetic operation unit as a result of the blood vessel being recognized at a plurality of different times.

### CROSS-REFERENCE TO RELATED APPLICATIONS

This is a continuation of International Application PCT/JP2016/062830 which is hereby incorporated by reference herein in its entirety.

This application is based on U.S. provisional patent application 62/151,585, the contents of which are incorporated herein by reference.

### TECHNICAL FIELD

The present invention relates to an endoscope apparatus.

### BACKGROUND ART

In surgical treatment of living tissue, it is important for an operator to accurately recognize the presence of a blood vessel hidden inside the living tissue and to perform treatment while avoiding the blood vessel. In response to this need, a surgical treatment device provided with a function for optically detecting a blood vessel that is present in living tissue has been proposed (refer to, for example, Patent Literature 1 below). According to Patent Literature 1, it is possible to measure the volume of blood in living tissue and to determine whether or not a blood vessel is present on the basis of the measured volume of blood, thus calling for the attention of the operator.

### CITATION LIST

### Patent Literature

### {PTL 1}

Publication of Japanese Patent No. 4490807

### SUMMARY OF INVENTION

One aspect of the present invention provides an image processing apparatus including: an image acquisition device for observing a living body; a blood vessel recognition device for irradiating the living body with an exploration laser beam and recognizing a blood vessel via a laser Doppler method; an arithmetic operation unit that calculates a first laser spot position on a first frame acquired by the image acquisition device when a blood vessel is recognized by the blood vessel recognition device and that adds a mark corresponding to the first laser spot position to a position on a second frame corresponding to the first frame, the second frame being acquired at a time different from the time of the first frame; and a display unit for displaying, on an image acquired by the image acquisition device, a plurality of the marks that are added by the arithmetic operation unit as a result of the blood vessel being recognized at a plurality of different times.

### BRIEF DESCRIPTION OF DRAWINGS

FIG. 1 is a diagram showing the overall configuration of an endoscope apparatus according to one embodiment of the present invention.

FIG. 2 is a diagram depicting scattering of a laser beam due to static components in living tissue.

FIG. 3 is a diagram depicting scattering of a laser beam due to dynamic components in living tissue.

FIG. 4 is a diagram depicting one example of time-series data of the intensity of scattered light acquired in a determination unit of the endoscope apparatus in FIG. 1.

FIG. 5 is a diagram depicting one example of a Doppler spectrum acquired in the determination unit of the endoscope apparatus in FIG. 1.

FIG. 6 is a diagram depicting the relationship between blood flow velocity and mean frequency of a Doppler spectrum.

FIG. 7 is a diagram depicting one example of the arrangement and the operation of a blood vessel recognition device and an image acquisition device in scope-assisted surgery.

FIG. 8 is a diagram depicting one example in which a displacement is acquired from a B-ch correlation image.

FIG. 9 is a diagram depicting one example in which the amount of displacement is calculated from a correlation image I_{Corr,B}(i+1).

FIG. 10 is a diagram depicting images observed without a laser beam.

FIG. 11 is a diagram depicting observation images when it is determined that there is no blood vessel (only an exploration laser beam is radiated).

FIG. 12 is a diagram depicting observation images when it is determined that there is a blood vessel (exploration and index laser beams are radiated).

FIG. 13 is a diagram depicting a case in which an image for spot position calculation is acquired and a spot position is calculated.

FIG. 14 is a diagram depicting a case in which the position where it is determined that there is a blood vessel is corrected for a displacement and is indicated with a mark.

FIG. 15A is a flowchart for illustrating image processing and display using the endoscope apparatus in FIG. 1.

FIG. 15B is a flowchart showing the continuation of the flowchart in FIG. 15A.

FIG. 15C is a flowchart showing the continuation of the flowchart in FIG. 15B.

FIG. 15D is a diagram for illustrating a specific example in the flowcharts of FIGS. 15A to 15C.

FIG. 15E is a diagram showing the continuation of FIG. 15D.

FIG. 15F is a diagram showing the continuation of FIG. 15E.

FIG. 16A is a flowchart for illustrating a modification of FIG. 15A.

FIG. 16B is a flowchart showing the continuation of the flowchart in FIG. 16A.

FIG. 16C is a flowchart showing the continuation of the flowchart in FIG. 16B.

FIG. 16D is a diagram for illustrating a specific example in the flowcharts of FIGS. 16A to 16C.

FIG. 16E is a diagram showing the continuation of FIG. 16D.

FIG. 16F is a diagram showing the continuation of FIG. 16E.

FIG. 17 is a diagram depicting a case in which an index laser beam is formed in the shape of cross-hairs and is calculated via image matching.

FIG. 18 is a diagram depicting a case in which an exploration laser beam is formed in a concentric-ring shape and is calculated via image matching.

FIG. 19 is a diagram depicting a correlation image based on images in which a laser spot undesirably intrudes.

FIG. 20 is a diagram of a modification of FIG. 19, depicting a correlation image (example of R-ch and B-ch) using images of other channels.

FIG. 21 is a diagram depicting a case in which a group of enlarged images, reduced images, and rotated images is generated and a correlation image is acquired.

FIG. 22 is a diagram depicting a case in which a group of affine transformation images is generated and a correlation image is acquired.

FIG. 23 is a diagram depicting a case in which the amount of shift is calculated from a feature point using the Lucas-Kanade method.

FIG. 24 is a diagram depicting the acquisition of the amount of shift using position sensors.

FIG. 25 is a diagram depicting a modification of FIG. 24.

FIG. 26 is a diagram depicting a modification of FIG. 7.

FIG. 27A is a flowchart for illustrating a modification of FIG. 15A.

FIG. 27B is a flowchart showing the continuation of the flowchart in FIG. 27A.

FIG. 27C is a diagram for illustrating a specific example in the flowcharts of FIGS. 27A and 27B.

FIG. 27D is a diagram showing the continuation of FIG. 27C.

FIG. 28A is a flowchart for illustrating a modification of FIG. 15A.

FIG. 28B is a flowchart showing the continuation of the flowchart in FIG. 28A.

FIG. 28C is a diagram for illustrating a specific example in the flowcharts in FIGS. 28A and 28B.

FIG. 28D is a diagram showing the continuation of FIG. 28C.

### DESCRIPTION OF EMBODIMENTS

An endoscope apparatus 200 according to one embodiment of the present invention includes: a blood vessel recognition device 100; an image acquisition device 500; an arithmetic operation unit 60; and a display unit 70.

The endoscope apparatus 200 will be described below with reference to the drawings.

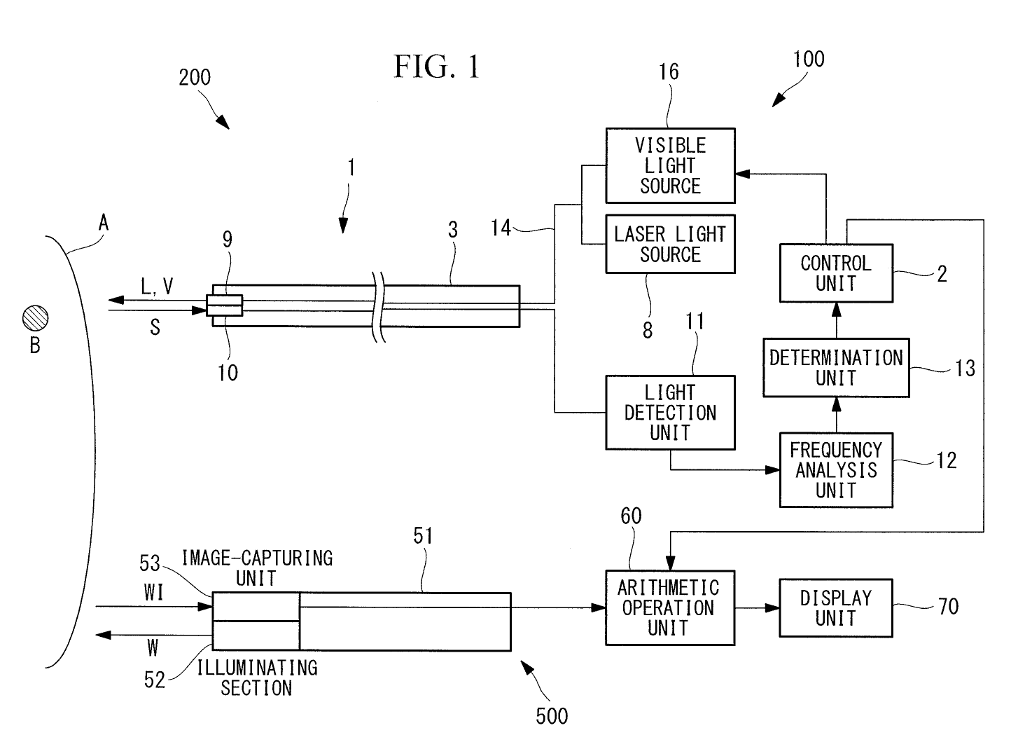

As shown in FIG. 1, the endoscope apparatus 200 includes: a treatment tool 1 provided with the blood vessel recognition device (blood vessel detecting means) 100 that optically detects a blood vessel B in living tissue A; a control unit 2 for controlling the outputting and stopping of visible light V from a light-emitting section 9 on the basis of a detection result from the blood vessel recognition device 100; the image acquisition device 500; the arithmetic operation unit 60; and the display unit 70. Here, the treatment tool 1 may be any device for performing surgery with the endoscope apparatus 200. The treatment tool 1 may be, for example, a device for performing incision of and arrest of bleeding in an affected area. Examples of such a device can include a device capable of outputting ultrasound energy and bipolar energy with a high-frequency current, and furthermore, an energy device for surgical treatment that is provided with a gripping tool for gripping an affected area may be provided at the leading end of the treatment tool 1.

The blood vessel recognition device 100 includes: an exploration laser light source 8 for outputting an exploration laser beam L; the light-emitting section 9 that is provided at a leading end of a probe body section and that emits the exploration laser beam L supplied from the exploration laser light source 8; a light receiving section 10 that is provided in the vicinity of the light-emitting section 9 and that receives scattered light S coming from the area in front of the leading end of the treatment tool 1; a light detection unit 11 for detecting the scattered light S received by the light receiving section 10; a frequency analysis unit 12 that acquires time-series data of the intensity of the scattered light S detected by the light detection unit 11 and that frequency-analyzes the time-series data; a determination unit 13 for determining the presence/absence of a blood vessel to be detected, which has a diameter in a predetermined range, on the basis of the frequency analysis result of the frequency analysis unit 12; and a visible light source 16.

The exploration laser light source 8 outputs the exploration laser beam L with a wavelength range that is barely absorbed into blood (e.g., near-infrared region). The exploration laser light source 8 is connected to the light-emitting section 9 via an optical fiber 14 that runs along the interior of a body section 3. The exploration laser beam L that is emitted from the exploration laser light source 8 and incident on the optical fiber 14 is guided to the light-emitting section 9 by the optical fiber 14 and is emitted towards the living tissue A from the light-emitting section 9. The exploration laser beam L radiated onto the living tissue A scatters in the interior of the living tissue but scatters differently depending on the presence/absence of a blood vessel.

The light receiving section 10 is, for example, a wavelength-selective photosensor for selectively outputting the amount of received light in response to the wavelength range of the exploration laser beam and is connected to the light detection unit 11 via an optical fiber 15 that runs along the interior of the body section 3. The scattered light S received by the light receiving section 10 is guided to the light detection unit 11 by the optical fiber 15 and is incident upon the light detection unit 11.

The light detection unit 11 converts, into a digital value, the intensity of the scattered light S that is incident thereon via the optical fiber 15 and sequentially transmits this digital value to the frequency analysis unit 12.

The frequency analysis unit 12 acquires the time-series data representing changes over time in the intensity of the scattered light S by recording the digital values received from the light detection unit 11 in a time series manner over a predetermined time period. The frequency analysis unit 12 applies fast Fourier transformation to the acquired time-series data and calculates the mean frequency of an obtained Fourier spectrum.

The image acquisition device 500 is composed of an endoscope body section 51, as well as an illuminating unit 52 and an image capturing unit 53 installed at a leading end section of the endoscope body section 51. White light W is radiated from the illuminating unit 52 towards the living tissue A. An observation image I of the living tissue A is acquired by acquiring an image with the image capturing unit 53 so as to detect observation image light WI resulting from the white light W.

Image information of the observation image I is sent to the arithmetic operation unit 60. A control signal from the control unit 2 of the blood vessel recognition device 100 is input to the arithmetic operation unit 60, and different arithmetic operations are performed on the basis of the control signal. Image information based on each arithmetic operation is added to the observation image I, and a display image I_{Disp}, is generated and displayed on the display unit.

Here, time-series data and a Fourier spectrum acquired in the image acquisition device 500 will be described.

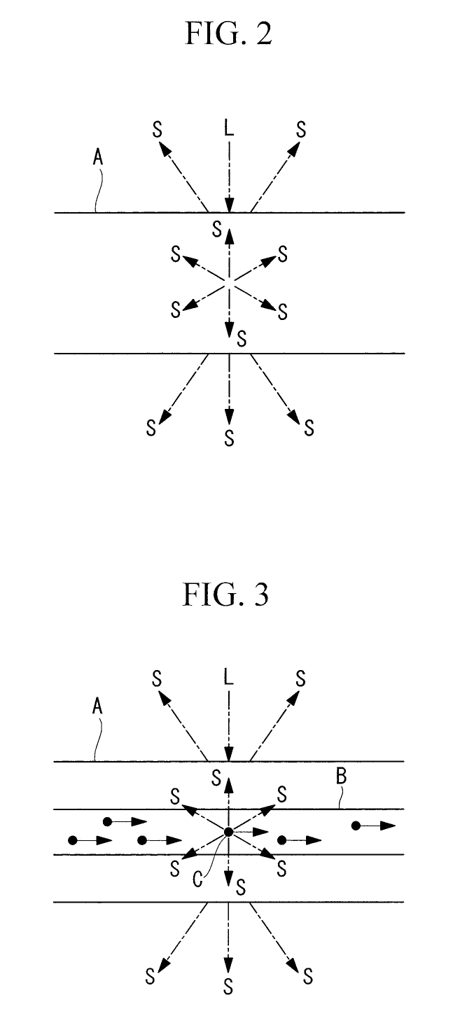

As shown in FIGS. 2 and 3, the living tissue A includes static components that are stationary, like fat and leaking blood exposed from a blood vessel due to bleeding, and moving dynamic components, like in-blood red blood cells C flowing in the blood vessel B. When the exploration laser beam L with a frequency f is radiated onto static components, scattered light S having the same frequency f as that of the exploration laser beam L is generated. In contrast, when the exploration laser beam L with a frequency f is radiated on dynamic components, scattered light S having a frequency f+Δf, shifted due to a Doppler shift from the frequency f of the exploration laser beam L, is generated. The amount of frequency shift, Δf, at this time depends on the velocity of the dynamic components.

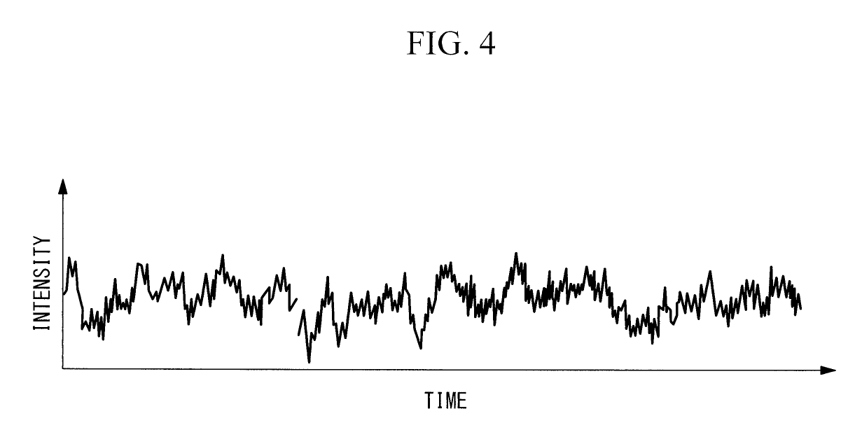

Therefore, when the blood vessel B is included in a radiation area of the exploration laser beam L in the living tissue A, scattered light S having a frequency f+Δf as a result of being scattered by the blood in the blood vessel B, as well as scattered light S having a frequency f as a result of being scattered by static components other than the blood in the blood vessel B, are simultaneously received by the light receiving section 10. This results in interference between the scattered light S with a frequency f and the scattered light S with a frequency f+Δf, leading to a beat causing the intensity of the entire scattered light S to change with Δf in the time-series data, as shown in FIG. 4.

Because the laser beam radiated on the living tissue A undergoes multiple scattering in the static components and dynamic components, when the laser beam is incident on the red blood cells, the incident angle between the traveling direction of the light and the moving direction of the red blood cells (blood flow direction) is not a single value but exhibits a distribution. Therefore, the amount of frequency shift, Δf, due to the Doppler shift also exhibits a distribution. Therefore, the beat of the intensity of the entire scattered light S includes superposition of multiple frequency components in accordance with the distribution of Δf. In addition, as the blood flow velocity becomes higher, the distribution of Δf extends to the higher frequency side.

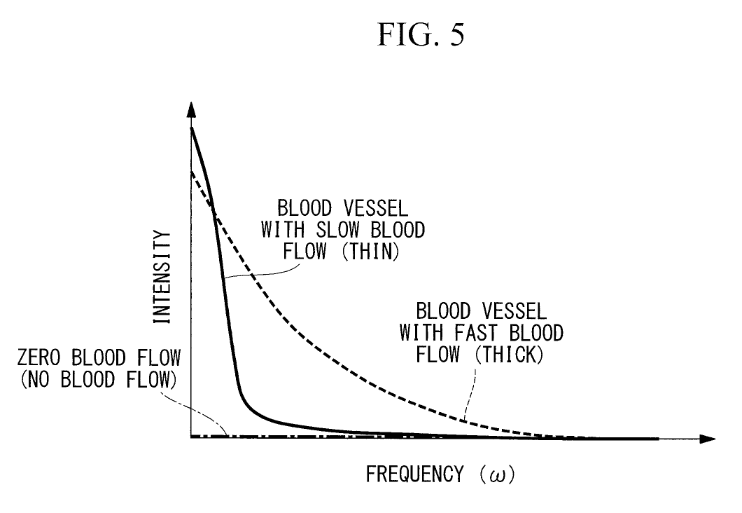

As shown in FIG. 5, when fast Fourier transformation is applied to such time-series data, a Doppler spectrum having an intensity at a frequency ω (hereinafter, the frequency shift Δf is referred to as ω) according to the blood flow velocity is obtained as a Fourier spectrum.

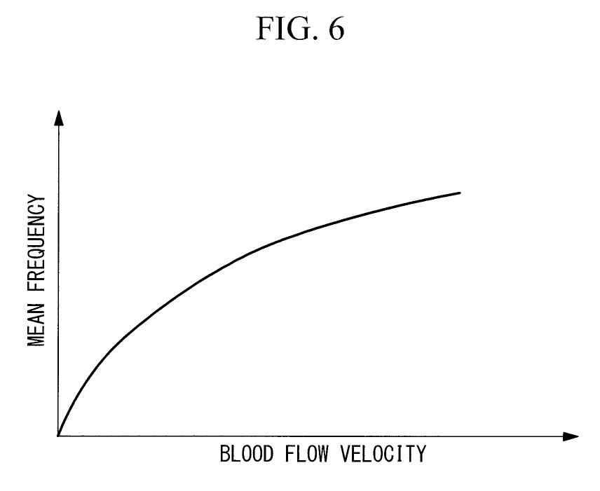

There is a relationship between the shape of the Doppler spectrum, and the presence/absence of the blood vessel B and the blood flow velocity in the blood vessel B, as shown in FIG. 5. There is a relationship between the mean frequency of the Doppler spectrum and the blood flow velocity, as shown in FIG. 6. More specifically, when the blood vessel B is not present in the radiation area of the exploration laser beam L, the above-described beat is not generated, and therefore, the Doppler spectrum becomes flat with no intensity in the entire region of the frequency ω (refer to the dotted-chain lines). When a blood vessel B with a slow blood flow is present, the Doppler spectrum has an intensity in a region near low frequencies ω and has a small spectral width (refer to the solid line). When a blood vessel B with a fast blood flow is present, the Doppler spectrum has an intensity in a region from a low frequency ω to a high frequency ω and has a large spectral width (refer to the dashed line). In this manner, the faster the blood flow, the farther the Doppler spectrum extends to the higher frequency ω side, and as the spectral width becomes larger, the mean frequency of the Doppler spectrum becomes higher.

Furthermore, it is known that the blood flow velocity in the blood vessel B is substantially proportional to the diameter of the blood vessel B.

The frequency analysis unit 12 obtains a function F(ω), representing the relationship between the frequency ω and the intensity of a Doppler spectrum, calculates the mean frequency of a Doppler spectrum F(ω) on the basis of expression (1) below, and transmits the calculated mean frequency to the determination unit 13.

$$Mean Frequency = \frac{\int ω F ( ω ) d ω}{\int F ( ω ) d ω} \qquad ( 1 )$$

The determination unit 13 compares the mean frequency received from the frequency analysis unit 12 with a threshold value. A first threshold value is the mean frequency corresponding to the minimum value of the diameter of the blood vessel B to be detected.

If the mean frequency received from the frequency analysis unit 12 is equal to or more than the first threshold value, then the determination unit 13 determines that the blood vessel B to be detected is present. On the other hand, if the mean frequency received from the frequency analysis unit 12 is less than the first threshold value, then the determination unit 13 determines that the blood vessel B to be detected is not present in the radiation area of the exploration laser beam L. By doing so, a blood vessel B having a diameter in a predetermined range is specified as a blood vessel to be detected, and it is determined whether or not this blood vessel B to be detected is present. The determination unit 13 outputs the determination result to the control unit 2 and the arithmetic operation unit 60.

The minimum value of the diameter of the blood vessel B to be detected is input by, for example, an operator using an input unit, which is not shown in the figure. For example, the determination unit 13 has a function for associating diameters of the blood vessel B with mean frequencies so as to obtain, from the function, the mean frequency corresponding to the minimum value of the diameter of the input blood vessel B and so as to set each of the calculated mean frequencies as a threshold value.

If it is determined by the determination unit 13 that the blood vessel B to be detected is present, then the control unit 2 causes the visible light V to be output from the visible light source 16, thereby emitting the visible light V from the light-emitting section 9 together with the exploration laser beam L. On the other hand, if it is determined by the determination unit 13 that the blood vessel B to be detected is not present, then the control unit 2 stops outputting the visible light V from the visible light source 16, thereby emitting only the exploration laser beam L from the light-emitting section 9.

Next, the operation of the endoscope apparatus 200 according to this embodiment with the above-described structure will be described.

An image display method using the endoscope apparatus 200 includes: a step of, if it is determined by the blood vessel recognition device 100 that a blood vessel is present (True), calculating a blood-vessel determination position on a measurement image from the measurement image; a step of calculating a displacement in the field of view by establishing a correlation between (arbitrary) two images in time series; a step of setting a mark position as the calculated position information on the basis of the displacement information; and a step of displaying a mark (trace) on a real-time image on the basis of the information about the mark position.

By doing so, even if the field of view shifts, the operator can be informed of where the blood vessel was located.

In short, the image display method according to this embodiment includes the following steps.
Step 0: Determines whether or not a blood vessel is present.
Step 1: Calculates a spot position on an image.
Step 2: Obtains the amount of shift in the field of view.
Step 3: Displays a position-corrected spot on the image where the field of view shifts.

One example for performing each of the above-described steps will be given below.

It is determined whether or not a blood vessel is present (step 0).

A spot position on an image at the time when an exploration laser beam and/or an index laser beam is radiated is calculated using at least one of methods (1) to (3) below (step 1).
(1) of step 1: A spot position is calculated by eliminating the sites saturated by white light by performing an AND operation between R and G-ch and the inversion of B-ch.
(2) of step 1: In the case of a complementary color optical system, a spot position is also calculated through the same processing as in (1) by converting the complementary color into RGB using a known method.
(3) of step 1: The laser spot is made to take a specific shape that differs from an Airy pattern, so that the laser spot is calculated via image matching. Here, the specific shape is a shape that is formed by combining a plurality of regular patterns and that can be recognized by the operator as a clearly different shape from a conventional shape of the exploration laser beam during exploration, while the shape still functions as a laser spot, and such a specific shape is realized, for example, (a) by radiating an index laser beam so as to take the shape of cross-hairs or (b) by radiating an exploration laser beam so as to take a concentric-ring shape.

The amount of shift in the field of view is obtained using methods (1) to (3) below (step 2).
(1) of step 2: Method for calculating the amount of shift through image processing applied to an inter-frame image. This method for calculating the amount of shift in (1) is performed with one of the means (a) and (b) below.
(a) Calculating the amount of shift by acquiring a correlation image.

A correlation image can be acquired using at least one of methods (a-1), (a-2), and (a-3) below.

A correlation image simply representing the image relationship between frames is acquired (a-1).

A group of enlarged images, reduced images, and rotated images is generated, and a correlation image is acquired (a-2). The ranges and steps of the enlargement, reduction, and rotation angle are not restricted and may be adjusted as appropriate.

A group of images obtained by affine transformation is generated, and a correlation image is acquired (a-3).
(b) Calculating the amount of shift using the Lucas-Kanade method.
(2) of step 2: Method in which a change in the position of a rigid endoscope (image acquisition device) 500, which is inserted as an insertion section into the abdominal cavity, is monitored and the amount of shift in the field of view is calculated from the change in the position.

Method (2) described above is performed using one of means (a) and (b) below.
(a) Detecting the relative position between a trocar 83 and the rigid endoscope 500 with insertion length/rotational position sensors (sensors) 84 and 85.
(b) Detecting the relative position using a position sensor (sensor) 86 mounted on the leading end of the rigid endoscope 500.
(3) of step 2: Combination of amount-of-shift calculation methods (1) and (2) above.

Instead of the spot before being corrected, a spot the position of which has been corrected with the amount of shift obtained in step 2 is displayed on the image where the field of view shifts (step 3).

According to the flow from step 0 to step 3 described above, even if the field of view shifts, a past blood vessel position after the amount of shift is corrected can be constantly displayed as an afterimage by calculating the position at which the blood vessel is recognized, correcting the amount of displacement, and displaying the shift-corrected position on the display unit, thereby making it possible to inform the operator of where the blood vessel was located on a real-time display screen.

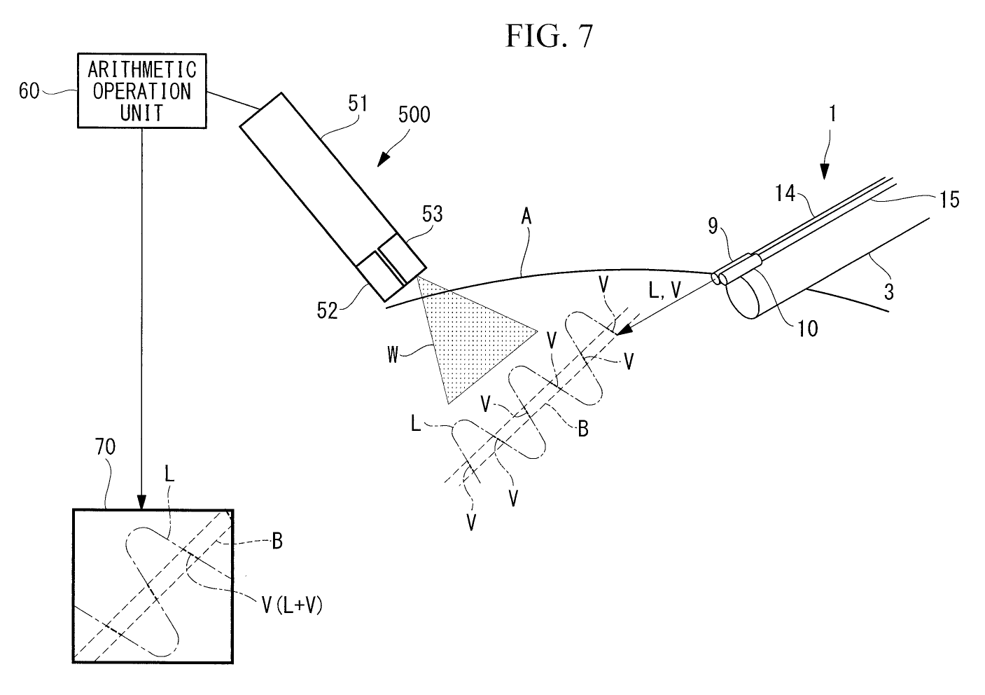

In order to recognize the blood vessel B in the living tissue A using the blood vessel recognition device 100 according to this embodiment, the light-emitting section 9 is placed in the vicinity of the living tissue A, the exploration laser beam L is radiated on the living tissue A, and the exploration laser beam L is moved so as to scan the living tissue A, as shown in FIG. 7. The scattered light S of the exploration laser beam L scattered in the living tissue A is received with the light receiving section 10.

If it is determined by the determination unit 13 that the blood vessel B to be detected is not present in the radiation area of the exploration laser beam L, the control unit 2 emits only the exploration laser beam L from the light-emitting section 9. If it is determined by the determination unit 13 that the blood vessel B to be detected is present in the radiation area of the exploration laser beam L, the control unit 2 emits the visible light V, together with the exploration laser beam L, from the light-emitting section 9. In short, this radiation area is irradiated with visible light V only when the blood vessel B to be detected is present in the radiation area of the exploration laser beam L.

A case in which the above-described situation is observed on an image acquired with the image acquisition device 500 will be described.

The region in which the blood vessel B is present can be recognized on the image displayed on a screen display unit by scanning the exploration laser beam L over the living tissue A and capturing, with an image capturing device, the visible light V radiated at the blood vessel position.

When a region in which the blood vessel is present can be identified with one scan operation, it is desirable that the region be held on the display screen since the operator can easily identify the region in which the blood vessel is present. However, because the endoscope body section 51 is not fixed under actual scope-assisted surgery, the image acquisition device 500 and the blood vessel recognition device 100 are configured to be able to move independently of each other, thereby readily causing a shift between the field of view acquired during scanning and the field of view that is being observed in real time.

Therefore, simply holding the scanning trajectory is useless when the field of view shifts.

In order to correct the shift in the field of view, it is advisable to calculate the displacement in the field of view by calculating a correlation between the two images in time series.

Here, a case in which a green laser beam is used as the visible light V will be described to explain the principle. Hereinafter, a description will be given assuming that the visible light V is an index laser beam V.

As described above, it is desirable that a laser beam with a wavelength (near-infrared region) that undergoes only a small amount of absorption/scattering in the living body be selected as the exploration laser beam L. At this time, if the R-ch of the image capturing unit 53 has sensitivity to the exploration laser beam L, the spot formed when the exploration laser beam is radiated on the living body is observed on the R-ch image of the image capturing unit 53.

In addition, the spot formed when the index laser beam V is radiated on the living body is observed on the G-ch image of the image capturing unit 53.

Because the proportion at which the spots of these laser beams contribute to the B-ch image is small, a displacement in the field of view is evaluated using the B-ch image.

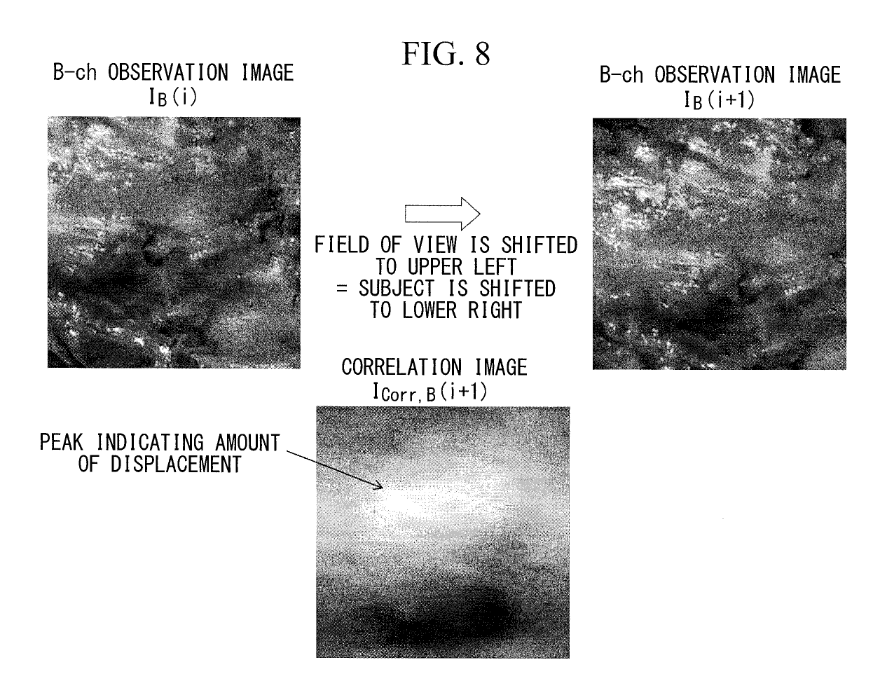

FIG. 8 shows images I_{B}(i) and I_{B}(i+1) of the B-ch in two frames i and i+1 being present in a certain time series. This figure shows a case in which the field of view moves to the upper left. In this figure, the subject moves to the lower right on the images. A correlation image I_{Corr,B}(i+1) formed by establishing a correlation between these images I_{B}(i) and I_{B}(i+1) is shown at the lower part. It is recognized that a peak appears on the correlation image I_{Corr,B}(i+1). This serves as an index of the amount of displacement as shown below.

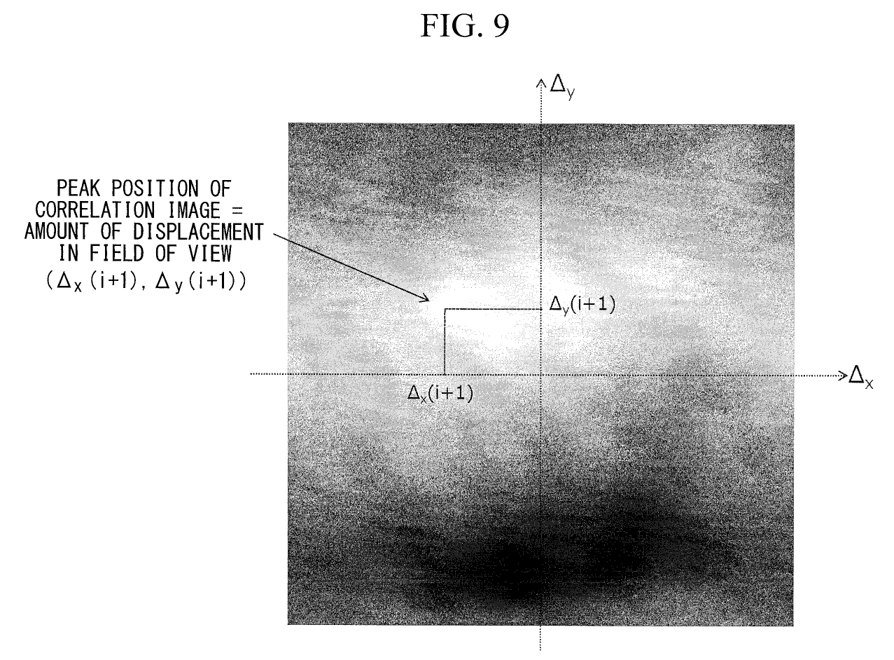

FIG. 9 shows the relationship between the peak and the displacement on the correlation image I_{Corr,B}(i+1). Each pixel on the correlation image is a value obtained by relatively displacing I_{B}(i) and I_{B}(i+1) on the Δx axis and the Δy axis and then calculating an overlap integral, and a correlation image is acquired by plotting integral values along the Δx axis and the Δy axis. Therefore, the Δx axis and the Δy axis are set so as to divide the correlation image longitudinally and laterally into equal sections, and the center of the image is the origin, which indicates displacement 0. The peak appearing on the correlation image indicates that the highest correlation is formed when the two images are overlapped with each other on the displacement at which the peak is positioned. In short, the peak position (Δx(i+1), Δy(i+1)) corresponds to the amount of displacement (Δx(i+1), Δy(i+1)) of the field of view.

Therefore, the amount of displacement (Δx(i+1), Δy(i+1)) can be obtained by calculating the peak position on the correlation image I_{Corr,B}(i+1).

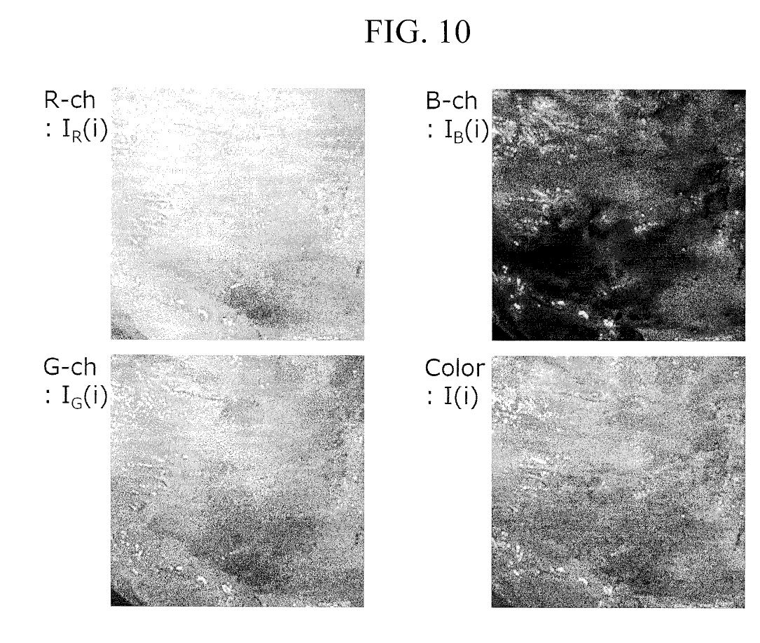

A method for calculating a blood vessel position from an image acquired with the image acquisition device 500 will be described below with reference to FIGS. 10 to 13.

FIG. 10 shows images observed when no laser beam is radiated. Observation images of R-ch, G-ch, and B-ch are denoted as I_{R}(i), I_{G}(i), and I_{B}(i), respectively, and an observation color image composed of them is denoted as I(i).

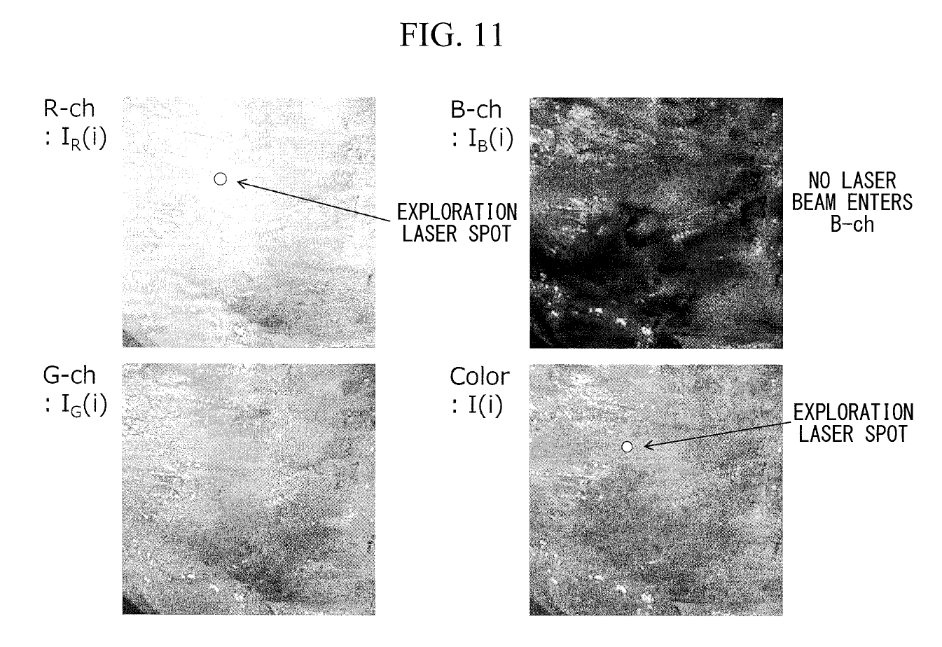

FIG. 11 shows observation images when there are no blood vessels. Because only the exploration laser beam is radiated at this time, the spot of the exploration laser beam is observed with high brightness only on I_{R}(i).

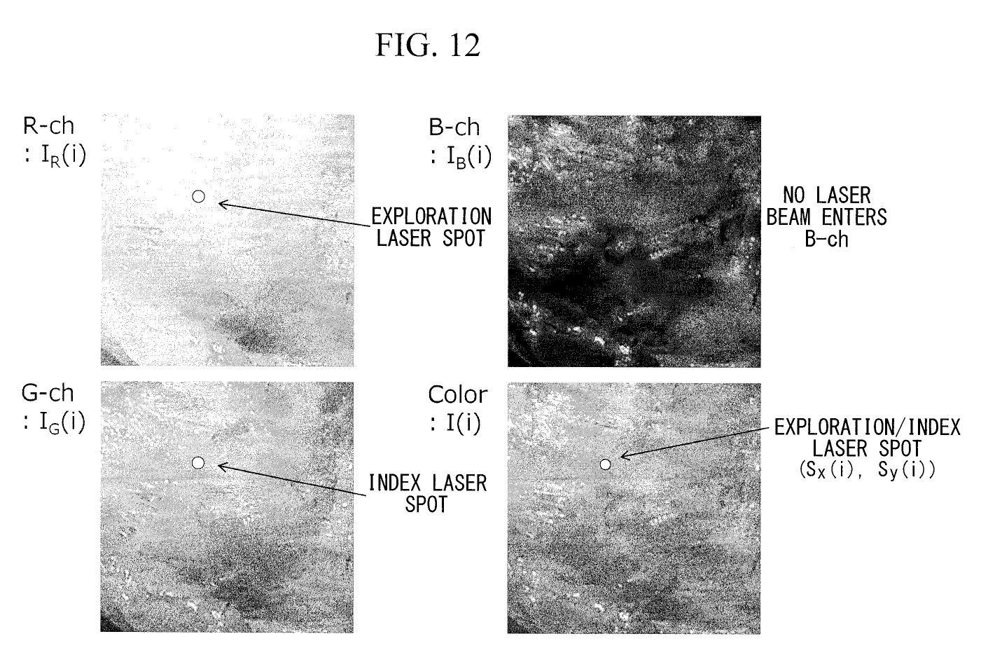

FIG. 12 shows observation images in a case where it is determined that a blood vessel is present. At this time, both the exploration and index laser beams L and V are radiated. In this case, although a laser spot is observed with high brightness on I_{R}(i) and I_{G}(i), no laser beam is observed on I_{B}(i).

In addition, it is recognized that the color image I(i) contains a region that appears white as a result of being saturated due to radiation of the white light W.

In order to accurately calculate the position irradiated with the laser beams from the observation image, this saturation region due to the white light W needs to be excluded.

Therefore, it is difficult to calculate a laser position only from brightness values of R/G-ch, and hence it is required to obtain a region that has high brightness on R/G-ch and that has low brightness on B-ch, thereby calculating a spot position from the center of the gravity of the region.

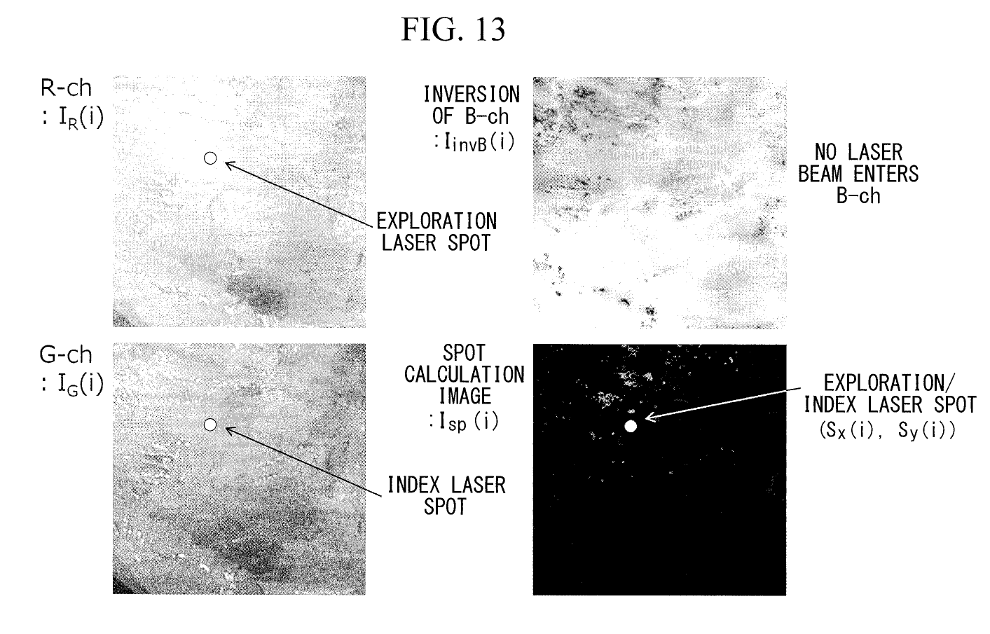

More specifically, in order to realize this, it is advisable to use the procedure shown in FIG. 13. In other words, an image I_{SP}(i) for spot calculation is acquired by using an inverted image B-ch and performing a logical add (AND) operation on I_{R}(i), I_{G}(i), and I_{invB}(i). By calculating the center of the gravity of a high brightness region on this I_{SP}(i), it is possible to calculate the spot position of the exploration/index laser beams, i.e., the position (Sx(i), Sy(i)) at which it is determined that there is a blood vessel. The acquired image I_{SP}(i) for spot calculation may be further adjusted to more appropriate contrast. In this manner, the accuracy of the position (Sx(i), Sy(i)) can be enhanced.

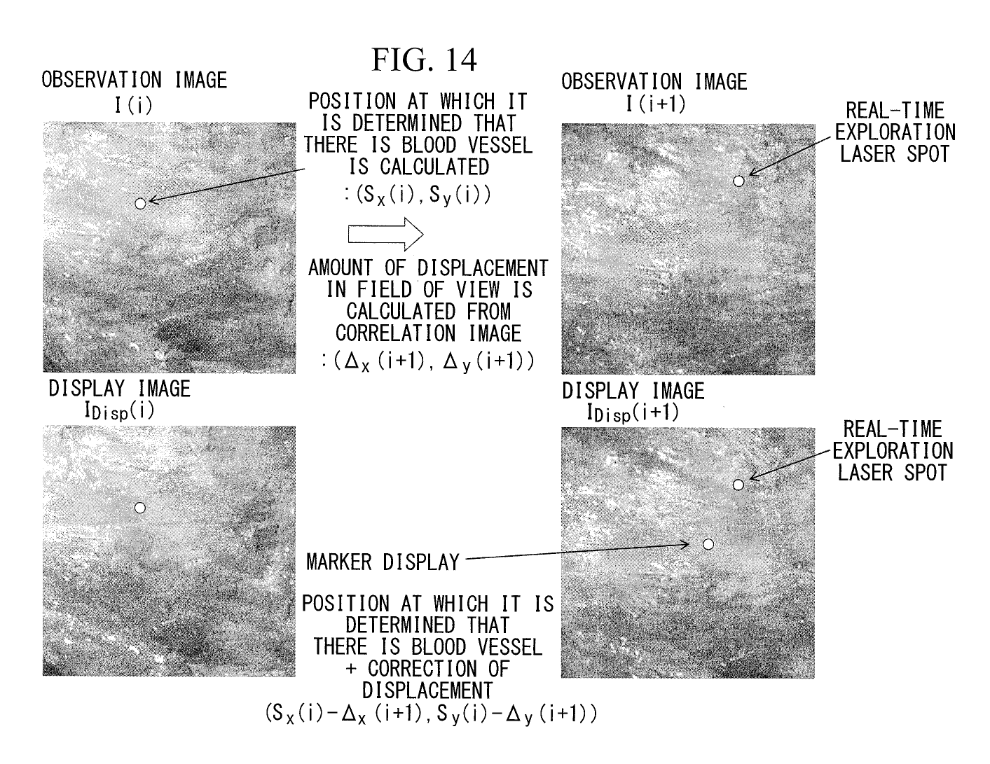

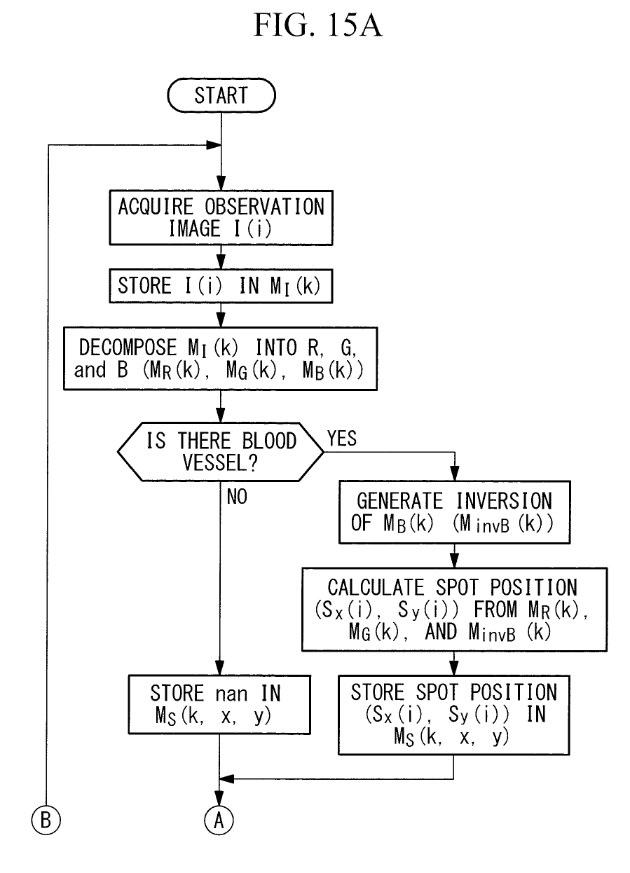

When it is determined that there is a blood vessel (True), the following processing is performed in the arithmetic operation unit 60, as shown in FIG. 14.
(1) Images I_{R}(i), I_{G}(i), and I_{B}(i) of each channel of RGB are acquired from the observation image I(i).
(2) I_{invB}(i) is acquired from I_{B}(i), a spot calculation image I_{SP}(i) is generated from I_{R}(i), I_{G}(i), and I_{invB}(i), and the position (Sx(i), Sy(i)) at which it is determined that there is a blood vessel is calculated.
(3) Images I_{R}(i+1), I_{G}(i+1), and I_{B}(i+1) of each channel of RGB are acquired from the observation image I(i+1) in frame i+1, which is located later in time series.
(4) The amount of field-of-view displacement (Δx(i+1), Δy(i+1)) is calculated from a correlation image I_{Corr,B}(i+1) between I_{B}(i) and I_{B}(i+1).
(5) A display image I_{Disp}(i+1) having a mark added at the position (Sₓ(i)−Δx(i+1), S_{y}(i)−Δy(i+1)) on the observation image I(i+1) at which it is determined that a blood vessel is present is generated by applying, as a correction value, the amount of displacement acquired in (4) above to the position (Sx(i), Sy(i)), so that the display image I_{Disp}(i+1) is displayed on the display unit 70 (final display state).

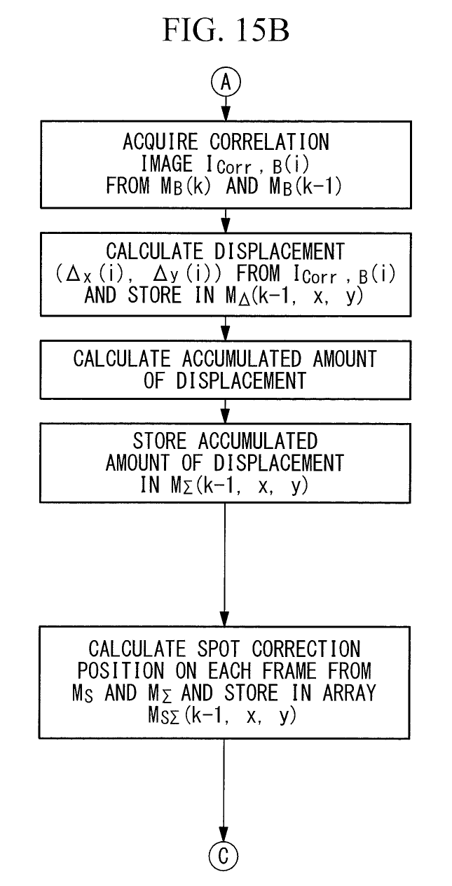

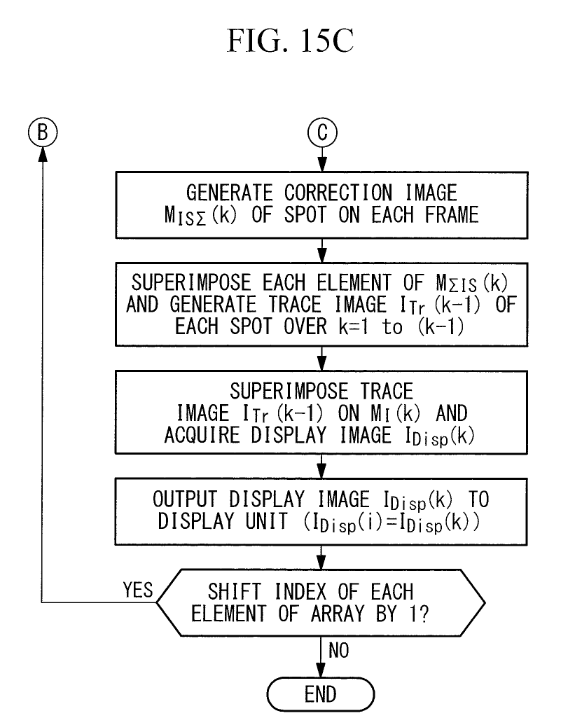

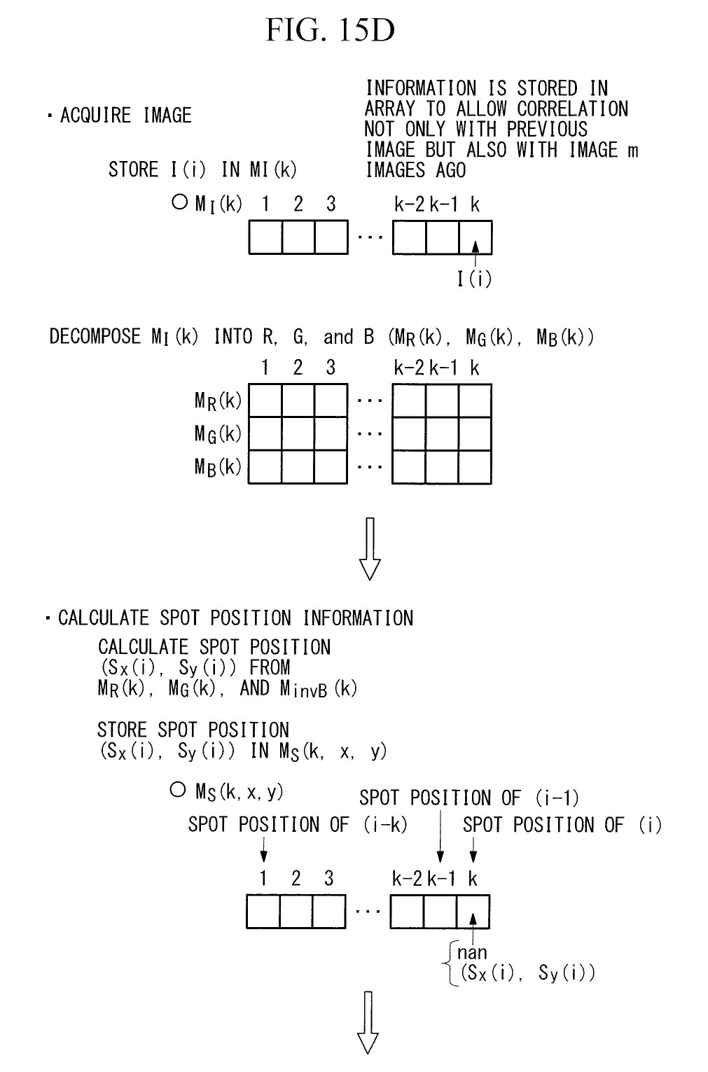

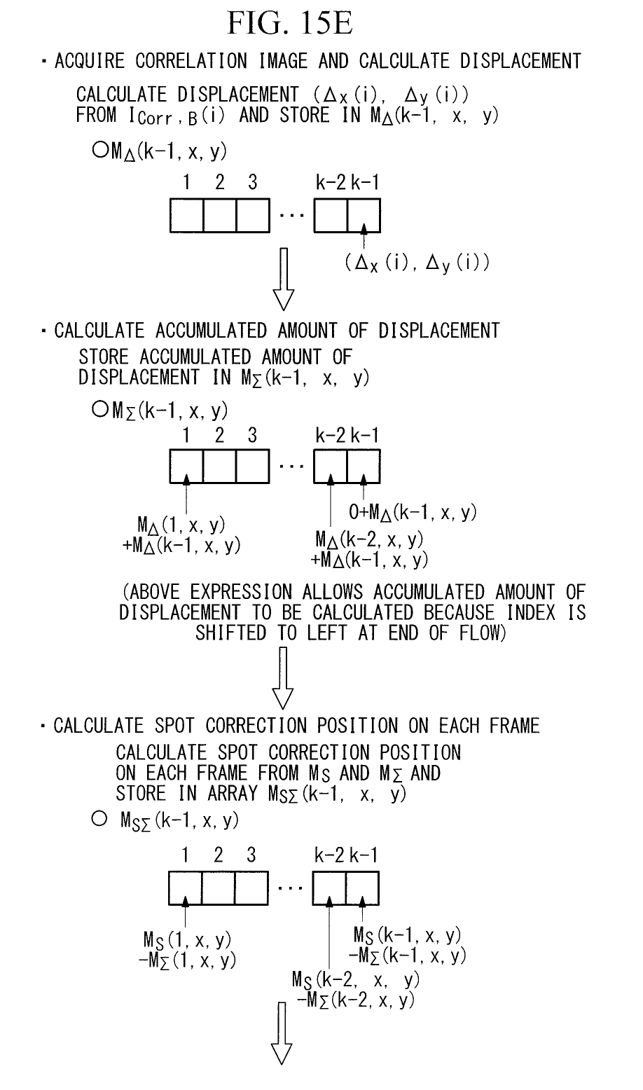

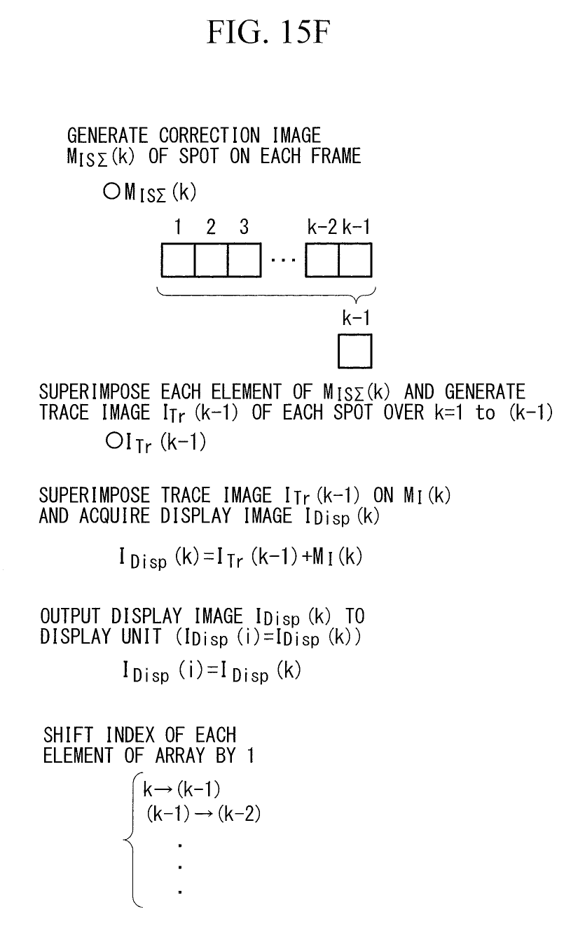

The problem can be solved by performing processing and display according to the flow in FIGS. 15A to 15F.

Flow (1) will be described below. First, an observation image I(i) is acquired in the i-th iteration.

This observation image I(i) is stored in the k-th element M_{I}(k) of an array M_{T}.

M_{I}(k) is decomposed into the channels R, G, and B to acquire M_{R}(k), M_{G}(k), and M_{B}(k).

Next, different processing is performed depending on the result of the blood vessel determination. First, if it is determined that there is a blood vessel (YES, i.e., True in the figure), then M_{invB}(k), which is the inversion of M_{B}(k), is generated, and the position at which it is determined that a blood vessel is present, i.e., the spot position (Sₓ(i), S_{y}(i)), is calculated from the three images M_{R}(k), M_{G}(k), and M_{invB}(k). This spot position (Sₓ(i), S_{y}(i)) is substituted into the k-th element M_{S}(k, X, Y) of an array M_{S}. Here, for the sake of convenience, the coordinates (Sₓ(k), S_{y}(k)) of the spot are denoted as M_{S}(k, X, Y), to indicate the k-th element of M_{S}. On the other hand, if it is determined that there are no blood vessels (NO, i.e., false in the figure), then nan, indicating that no corresponding spot position is present, is substituted into the k-th element M_{S} (k, X, Y) of the array M_{S}.

Next, displacement is calculated.

A correlation image I_{Corr,B}(i) is acquired on the basis of the images M_{B}(k) and M_{B}(k−1).

The displacement (Δx(i), Δy(i)) of measurement (i) relative to the measurement (i-1) is calculated from the correlation image i_{Corr,B}(i) and is stored in an array M_{A}(k−1, x, y) of displacements.

Subsequently, the accumulated amount of displacement, is calculated from each element. The accumulated amount of displacement is obtained by adding the displacement M_{Δ}(k−1, x, y) to the amount of displacement M accumulated up to that point. Note that all elements are set to 0 as the initial values of M_{Σ}.

M_{Σ}(k−1, x, y)=0+M_{Δ}(k−1, x, y), M_{Σ}(k−2, x, y)=M_{Δ}(k−2, x, y)+M_{Δ}(k−1, x, y), . . . M_{Σ}(1, x, y)=M_{Δ}(1, x, y)+M_{Δ}(k−1, x, y)

Note that because processing for shifting the index, indicating each array element, to the side smaller by 1 is performed at the end of the loop, the smaller the index becomes, such as k−2, k−3, . . . 1, the accumulated displacement reflects that the amounts of displacements on images of earlier frames have been added.

Next, the accumulated amount of displacement is added to the spot position obtained on each frame (at the time of each measurement) and is stored in an array M_{Sσ}(k−1, x, y).

A correction image M_{ISΣ}(k) is produced by placing a mark of a desired size on the image of each frame on the basis of this array information. The size of the mark may be the size in accordance with the actual spot diameter, or an image made to have a hue that is easy to identify is also acceptable.

By superimposing each element of these correction images M_{ISΣ}(k), a trace image I_{Tr}(k−1) of the marks over k=1 to (k−1) is generated. Here, if k is 2, then a single mark is produced, and if k>2, then the operation of superimposing a plurality of marks is performed, and therefore, a trace image of the spot positions (marks) on the frames is obtained.

Next, by superimposing the trace image I_{Tr}(k−1) on the M_{T}(k), i.e., observation image I(i), a display image I_{Disp}(k) is acquired.

The display image I_{Disp}(k) is output to the display unit 70 (I_{Disp}(i)=I_{Disp}(k) in the case of measurement i).

The index indicating each element of array data is shifted to the side smaller by one at the end of the processing loop. By doing so, information acquired on the previous frame is accumulated.

The next observation image I(i+1) is acquired via the loop, and the same processing is continued.

The length of the accumulated information is specified by setting the size k of the array as appropriate, and hence the time period for which the trace image of blood vessel detection positions is displayed can be set.

It is preferable that an ON/OFF button for turning ON/OFF laser beam radiation for blood vessel recognition be provided so as to allow the operation of blood vessel recognition to be temporarily interrupted, for cases in which the treatment tool 1 provided with the blood vessel recognition device 100 and/or the endoscope apparatus 200 is moved to another affected area or observation field of view and blood vessel recognition is resumed or for cases in which only the operation for treatment of the affected area with the treatment tool 1 is performed before/after or during the operation of blood vessel recognition. By doing so, even if a surgery assistant secures a stable field of view, display of a trace image with low correlation can be prevented by setting the ON/OFF button to OFF in a case where the treatment tool 1 provided with the blood vessel recognition device 100 and/or the endoscope apparatus 200 is moved for purposes other than blood vessel recognition. When laser beam radiation is set ON again, the above-described flow in FIGS. 15A to 15 F can be resumed by allowing the endoscope apparatus 200 to detect the laser beam radiation position. In addition, the trace image to be displayed in the loop does not need to display all information based on the measured image. By doing so, the processing speed can be enhanced, and furthermore, display becomes more visually recognizable by eliminating excessive trace images. On the other hand, in a case where three or more trace images are tilted at a certain angle relative to one another in a region where a plurality of different blood vessels run, the apparent resolution may be enhanced by displaying the marks so as to be connected to one another.

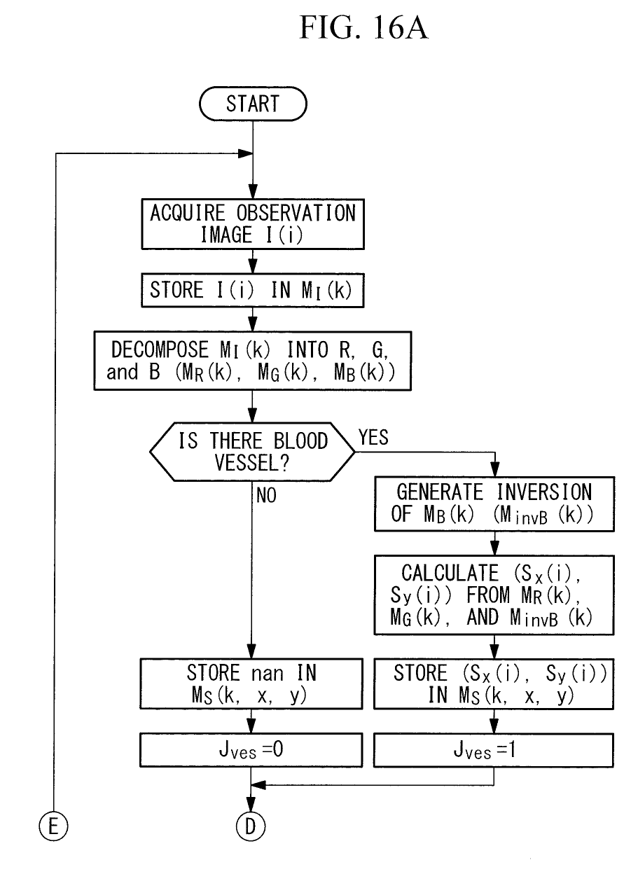

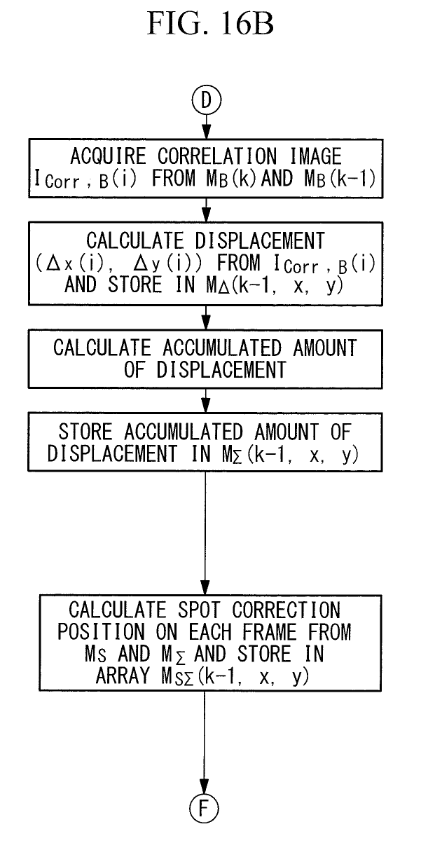

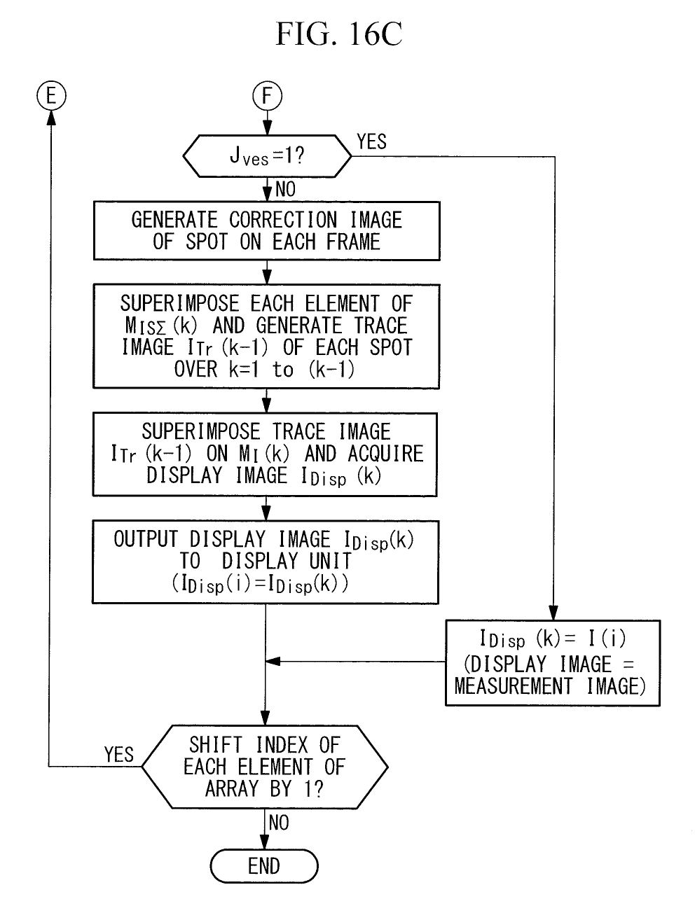

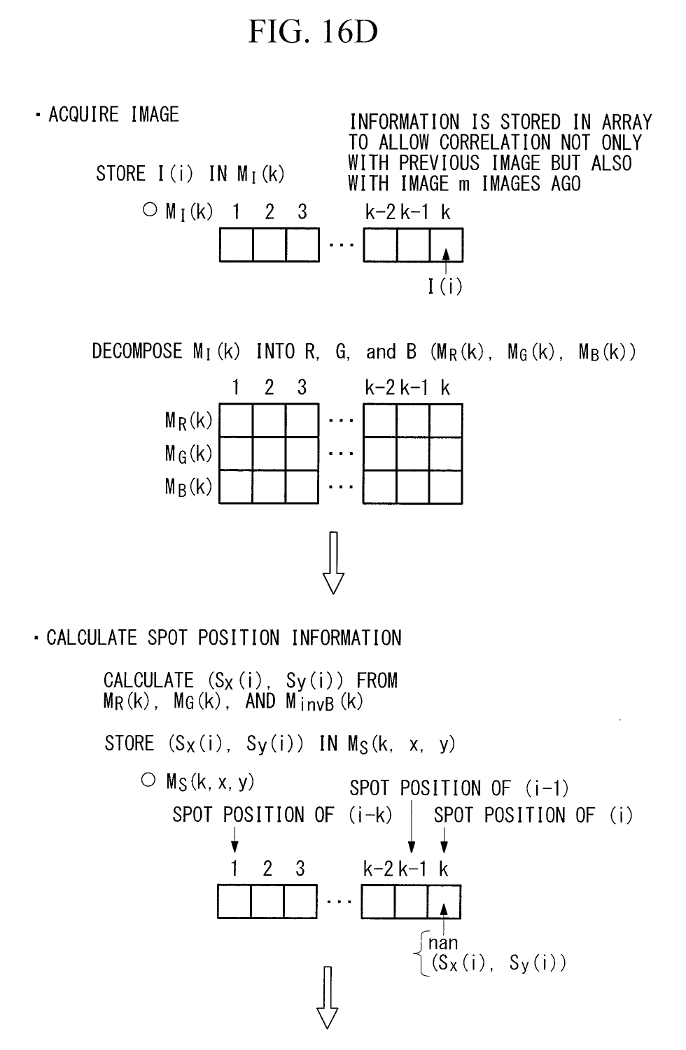

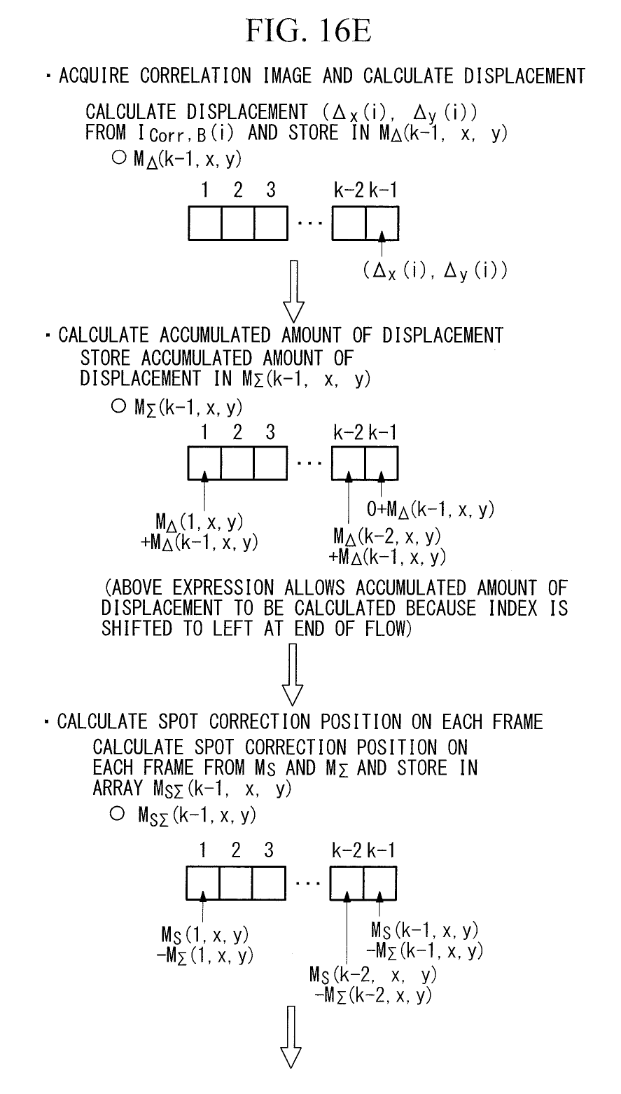

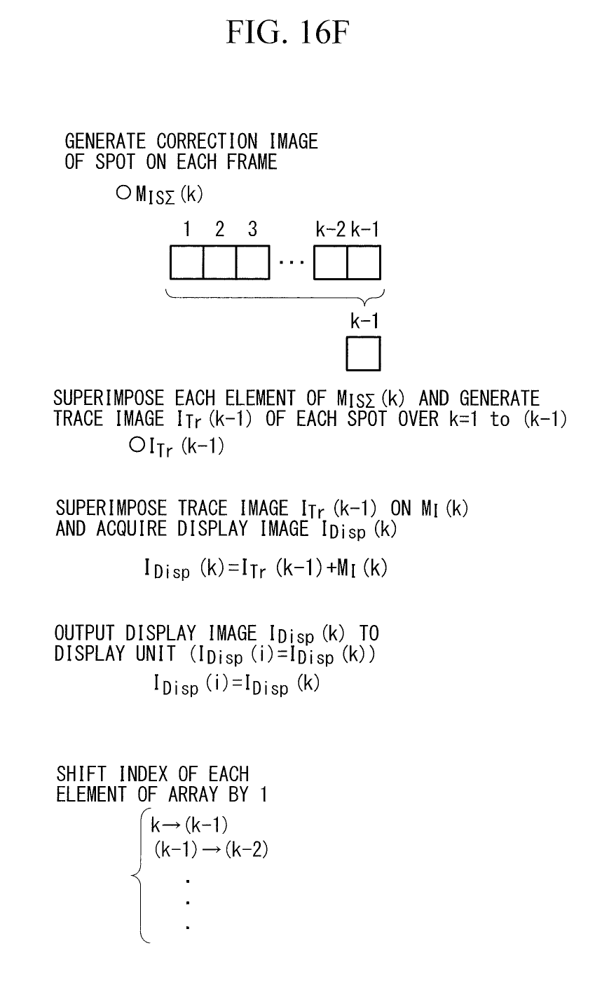

Although flow (2) in FIGS. 16A to 16F is identical to flow (1) in FIGS. 15A to 15F, 1 is substituted into a determination constant Jᵥₑₛ in the case of YES, and 0 is substituted into the determination constant Jᵥₑₛ in the case of NO after blood vessel recognition is performed.

Furthermore, a trace is displayed only when the determination constant Jᵥₑₛ is 0 downstream of the flow. This affords an advantage in that it is easy to identify a real-time determination spot because the historical record of determination is not superimposed while a determination that there is a blood vessel is maintained.

In the case of the complementary color optical system, it is possible to calculate a spot position on the image by performing the same processing as in step 1.1 using information obtained by conventional RGB conversion.

For (2) of step 1, processing can be performed in the same manner as in (1) of step 1 using information about RGB conventionally converted in the complementary color optical system.

For example, image processing of the complementary color optical system (refer to the Publication of Japanese Patent No. 4996773) is performed.

First, the following processing is performed:

Magenta (Mg), green (G), cyan (Cy), yellow (Ye)

↓Y/C separation circuit

Luminance signal Y, color-difference signal Cr′, Cb′

↓

R1, G1, B1

Thereafter, processing can be performed by using the images obtained with these signals for I_{R}(i), I_{G}(i), and I_{B}(i) in step 1.

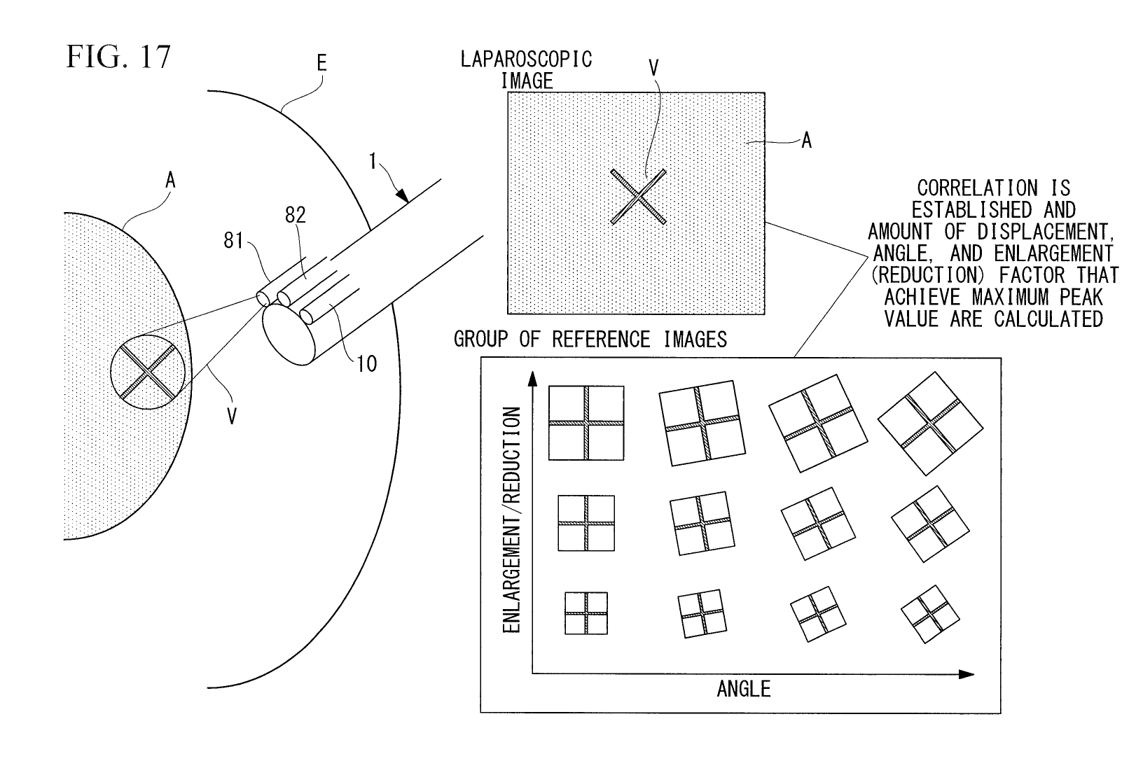

As (3)(a) of step 1, a spot position may be calculated by causing the index laser beam V to take the shape of cross-hairs, as shown in FIG. 17, and then performing image matching.

When an inverse Fourier image in the shape of cross-hairs is placed on the surface of the lens mounted on an emission unit 81 for the index laser beam V and a laser beam is radiated, a laser spot in the shape of cross-hairs is projected on a sample.

This spot in the shape of cross-hairs is observed on a laparoscopic image.

Correlations between a group of reference images, which are prepared to contain cross-hairs having sizes (enlarged/reduced) and angles shifted little by little relative to one another, and the above-described observed laparoscopic image are established, and the correlation image having the maximum peak value is selected. A spot position is calculated from the coordinates of the central portion of the cross-hairs.

The spots in the shape of cross-hairs have shapes of lines intersecting each other at substantially uniform angles, and hence, even in a case where the index laser beam V leaks not only into the B-ch but also into another ch, a spot position can be accurately calculated by differentiating an overexposed white spot from the index laser spot on an image via pattern matching of the specific shape, which is not present in the living body. Although, in this embodiment, the index laser beam V is made to exhibit a spot in the shape of cross-hairs composed of two orthogonal straight lines, the index laser beam V may be made to exhibit a spot in the shape of cross lines composed of three or more intersecting lines, like those composed of two or more lines intersecting at uniform angles.

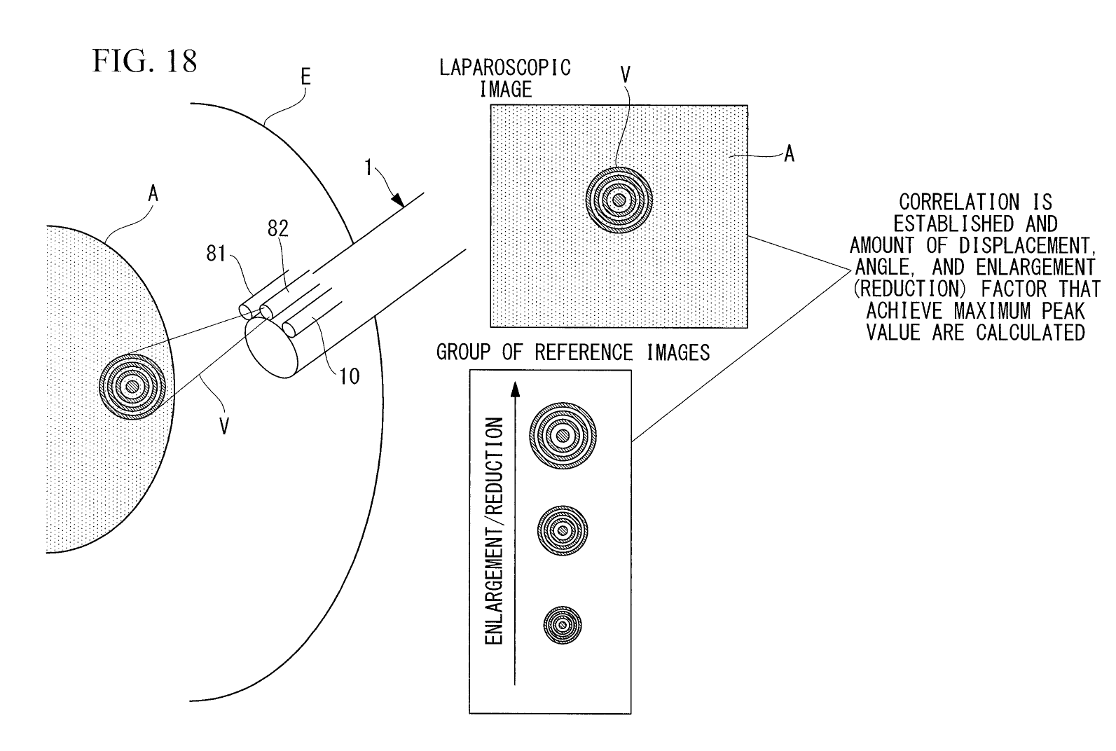

As (3)(b) of step 1, an embodiment in which the spot of the exploration laser beam L has a concentric-ring shape will be described with reference to FIG. 18.

When a laser beam is radiated from an emission unit 82 for the exploration laser beam L, a spot in a concentric-ring shape is observed on the laparoscopic image.

Correlations between a group of reference images, which are prepared to contain images of spots in a concentric-ring shape shifted little by little relative to one another, and the above-described observed laparoscopic image are established, and the correlation image having the maximum peak value is selected. For example, as shown in FIG. 18, a spot position is calculated from the coordinates at the spot center of the small circle provided at the central portion of a ring-shaped spot composed of multiple concentric circles with different sizes.

As a result of using a spot in a concentric-ring shape, even in a case where the index laser beam V leaks not only into the B-ch but also into another ch, a spot position can be accurately calculated by differentiating an overexposed white spot from the index laser spot on an image via pattern matching of the specific shape, which is not present in the living body.

Compared with the case where a spot in the shape of cross-hairs is used, this embodiment affords an advantage in that the group of reference images contains a smaller number of images because this group does not require the variable representing the angle direction, thus enhancing the processing speed at the time of exploration. Although, in this embodiment, the exploration laser beam L is made to exhibit a spot composed of a plurality of rings arranged on concentric circles, a concentric-ring shaped spot formed of a spiral having a known curvature is also acceptable.

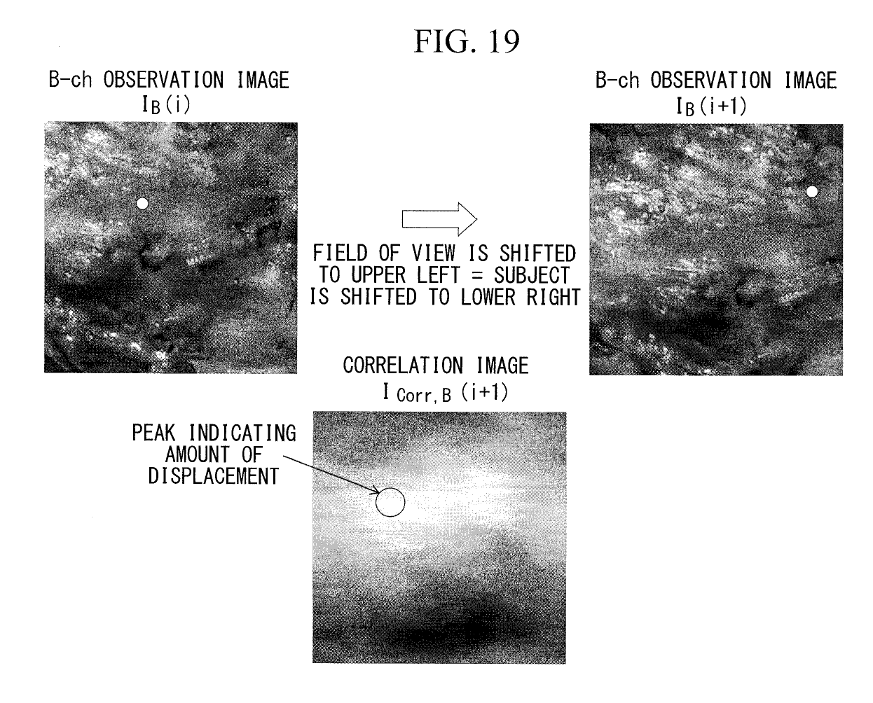

As a modification of (1) (a-1) of step 2, an example in which the amount of displacement is calculated using a correlation image based on images in which a laser spot undesirably intrudes will be described with reference to FIG. 19.

Here, a case in which a laser spot is contained (a case of undesirable intrusion thereof) in the B-ch image is described. The laser spot is contained at different positions in the i-th frame I_{B}(i) and the (i+1)-th frame I_{B}(i+1). On the correlation image I_{Corr,B}(i+1) between these images, a peak indicating a displacement is also observed. The displacement can be obtained by calculating the position of this peak.

Therefore, the displacement can also be calculated using an image in which a laser spot undesirably intrudes.

Although a filter with a high OD value needs to be used in order to prevent undesirable intrusion of a laser spot, this embodiment affords an advantage in that a displacement can be calculated as described above without having to replace the filter.

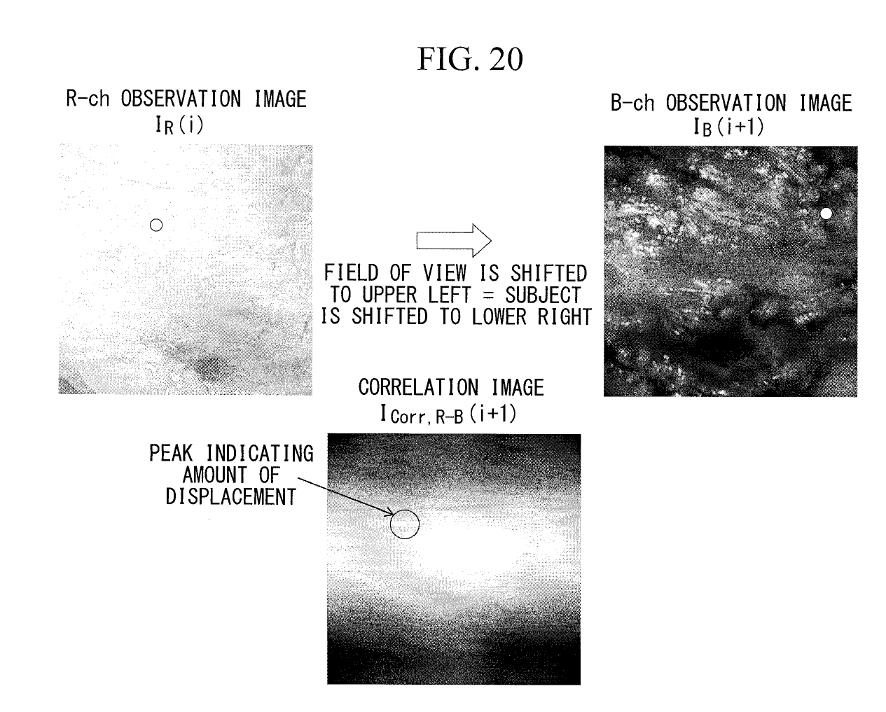

As a modification of (1) (a-1) of step 2, an example in which a spot position is calculated on the basis of a correlation image using images of other channels will be described with reference to FIG. 20.

Here, an example in which a laser spot is contained at different positions in the i-th frame I_{R}(i) of the R-ch and the (i+1)-th frame I_{B}(i+1) of the B-ch is given. On the correlation image I_{corr,R-B}(i+1) between these images, a peak indicating a displacement is also observed. This peak sharply rises compared with the peak at the center. Therefore, this peak can be enhanced by performing image processing using a high-pass filter that removes changes having a low frequency, consequently making it possible to calculate the peak position. A displacement can be calculated from this peak position.

Therefore, a displacement can be calculated by acquiring a correlation image between images of other channels.

This embodiment affords an advantage in that arbitrary images can be used according to the processing speed.

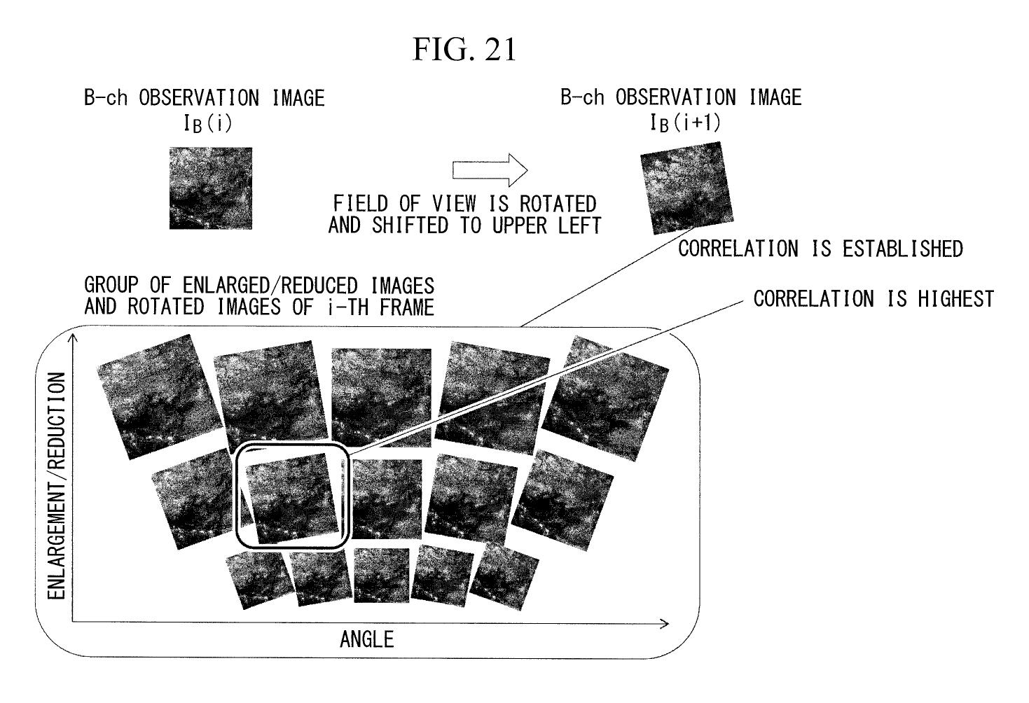

As (1)(a-2) of step 2, a method for acquiring a correlation image by generating a group of enlarged images, reduced images, and rotated images will be described with reference to FIG. 21.

The figure shows an example in which the field of view on the i+1-th frame is rotated and moved to the upper left relative to the i-th frame.

If rotation or enlargement takes place in addition to parallel translation in this manner, a group of images formed by enlarging, reducing, and rotating the i-th frame is generated, and a correlation between this group and the (i+1)-th frame is established. Each of the constituent components of the image group is endowed with information about an enlargement factor and a rotational angle. Of the correlation images, the correlation image with the maximum peak (image with the maximum correlation) is selected, and the amount of displacement is calculated. Furthermore, how much the i+1 -th field of view has been subjected to enlargement/reduction and rotation can be obtained from the enlargement factor and the rotational angle of the component of the image group that has generated the correlation image with the maximum peak.

On the basis of the above-described information, the amount of shift in spot position is obtained.

In this manner, the amount of shift in spot position can be calculated even in the case where the field of view is subjected to displacement, enlargement/reduction, and rotation.

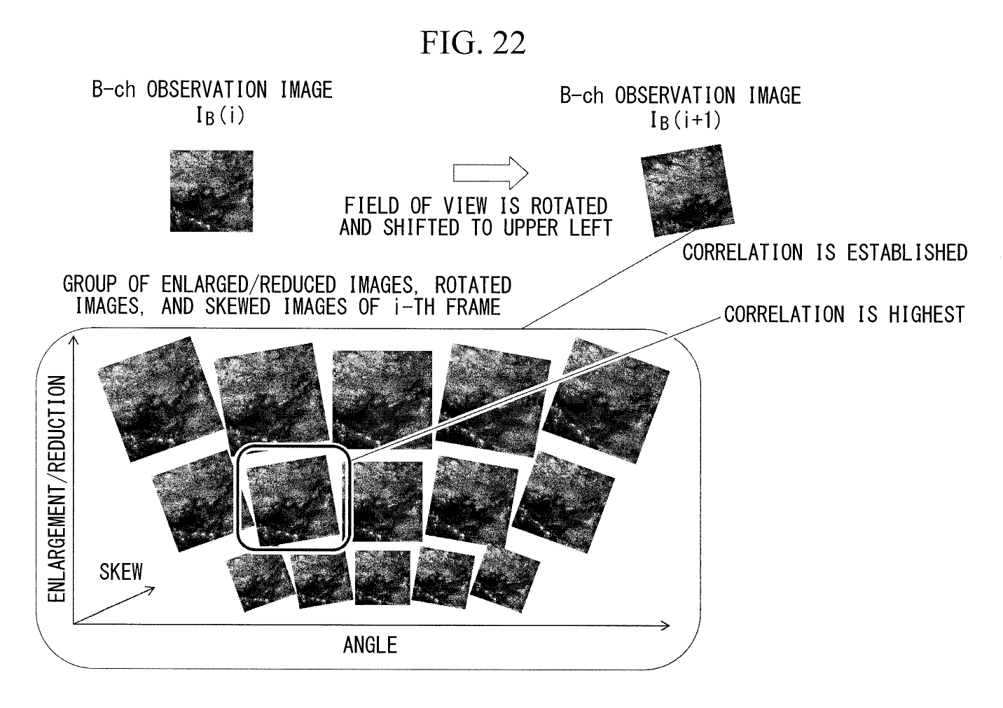

As (1)(a-3) of step 2, a method for acquiring a correlation image by generating a group of affine transformed images will be described with reference to FIG. 22.

The figure shows an example in which the field of view on the i+1-th frame is rotated and moved to the upper left relative to the i-th frame.

If rotation or enlargement takes place in addition to parallel translation in this manner, a group of images formed by applying affine transformation to the i-th frame is generated, and a correlation between this group and the (i+1)-th frame is established. Affine transformation includes enlargement/reduction, angle transformation, and skew transformation. Each of the constituent components of this image group is endowed with information about an enlargement factor, a rotational angle, and a skew. Of the correlation images, the correlation image with the maximum peak (image with the maximum correlation) is selected, and the amount of displacement is calculated. Furthermore, how much the i+1-th field of view has been subjected to enlargement/reduction, rotation, and skew can be obtained from the enlargement factor, the rotational angle, and the skew information of the component of the image group that has generated the correlation image with the maximum peak.

On the basis of the above-described information, the amount of shift in spot position is obtained.

In this manner, the amount of shift in spot position can be calculated even in the case where the field of view is subjected to displacement, enlargement/reduction, rotation, and skew.

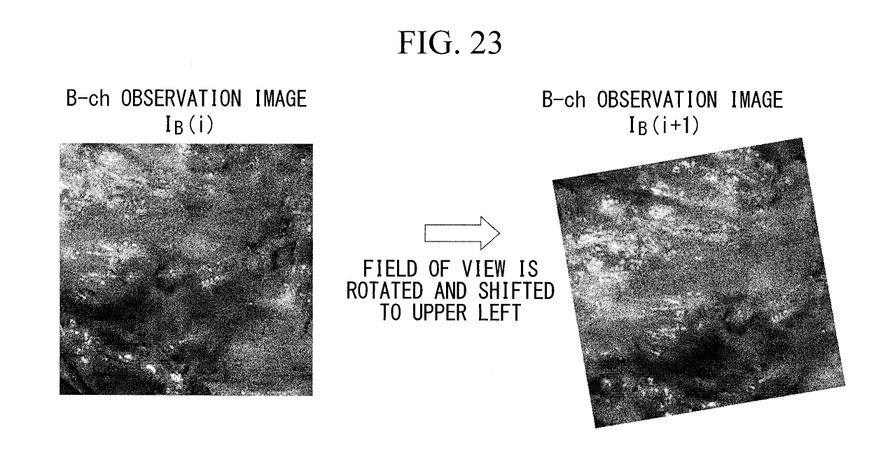

As (1)(b) of step 2, an example in which the amount of shift is calculated from a feature point obtained by the Lucas-Kanade method, as shown in FIG. 23, is given.

In this manner, the amount of shift in spot position can be calculated even in the case where the field of view is subjected to displacement, enlargement/reduction, and rotation.

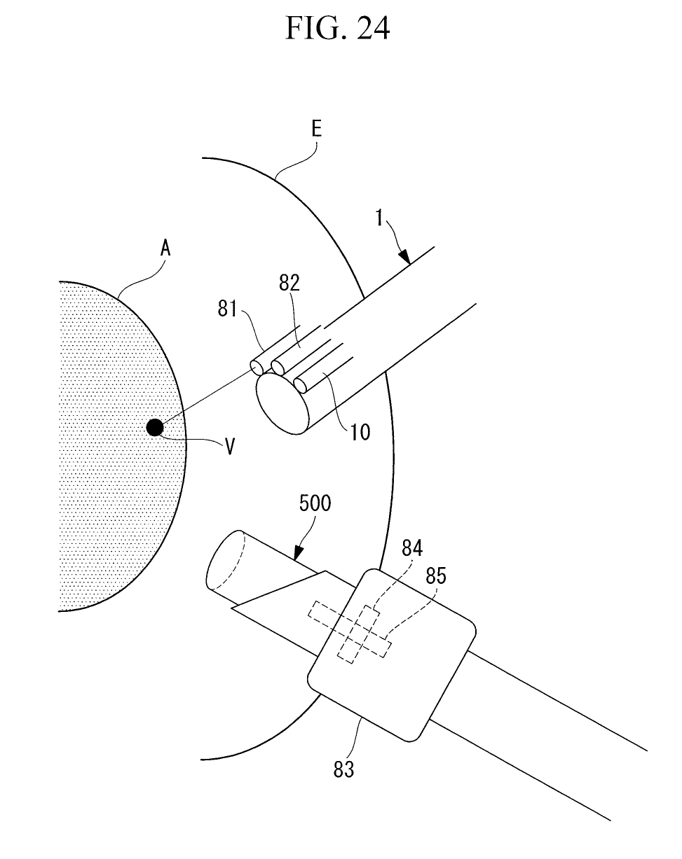

As (2)(a) of step 2, an embodiment in which the amount of shift is acquired with the insertion length/rotational position sensors 84 and 85 will be described with reference to FIG. 24.

In this embodiment, the amount of relative movement of the trocar 83 is calculated with the rotational position sensor 84 and the insertion length sensor 85 installed between the rigid endoscope 500 and the trocar 83.

Because how much the rigid endoscope 500 has moved towards or away from the living sample is known from the insertion length, the relative enlargement/reduction factor between frames can be calculated.

In addition, the amount of relative rotation between frames can be calculated with the rotational position sensor 84.

A spot is displayed at the corrected position on the basis of the amount of this shift (enlargement/reduction factor and amount of rotation).

A position shift can be calculated more accurately by using the sensors.

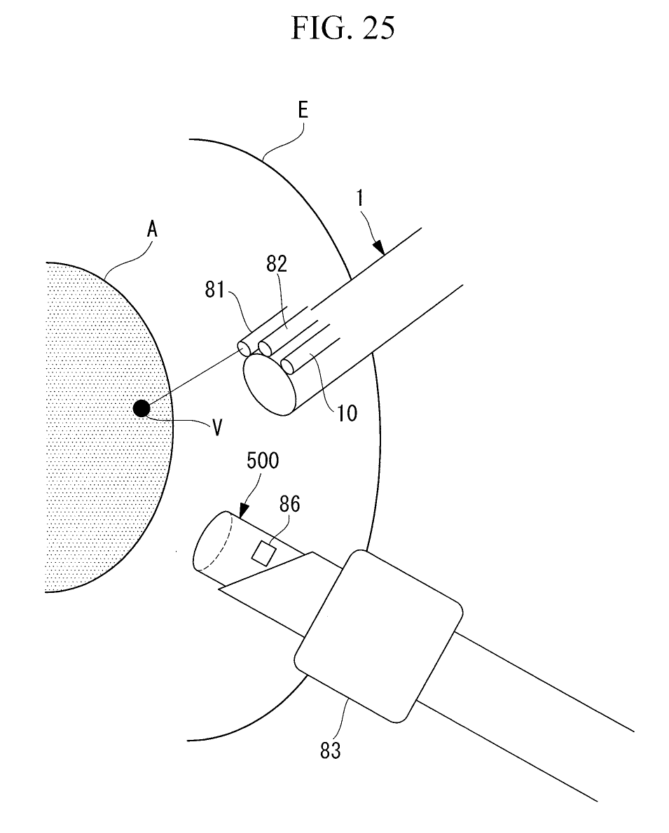

As (2)(b) of step 2, an embodiment in which the amount of shift is acquired with the electromagnetic position sensor 86 will be described with reference to FIG. 25.

The amount of relative movement (the amount of shift) of the rigid endoscope 500 is acquired by placing the electromagnetic position sensor 86 at the leading end section of the rigid endoscope 500 and acquiring position information with an electromagnetic detector, which is not shown in the figure.

By doing so, the amount of shift can be acquired without having to install a new sensor on the trocar 83 itself.

As (3) of step 2, (1) acquisition of the amount of shift via image processing may be combined with (2) acquisition of the amount of shift with a sensor.

By doing so, the amount of shift can be calculated more accurately.

Calculation of the amount of shift in this embodiment is not limited to the calculation methods described in the description. Instead, other known calculation methods may be used. For example, in (3) of step 1, the index laser beam in (a) may be made to exhibit a spot in a concentric-ring shape, instead of the shape of intersecting lines (e.g., cross-hairs), and furthermore, the exploration laser beam in (b) may be made to exhibit a spot in the shape of intersecting lines (e.g., cross-hairs), instead of a concentric-ring shape.

In addition, although this embodiment has been described by way of an example of the endoscope apparatus 200 in which the blood vessel recognition device 100 and the image acquisition device 500 are independent of each other, the endoscope apparatus 200 is not limited to this. Instead, the blood vessel recognition device 100 and the image acquisition device 500 may be integrated with each other.

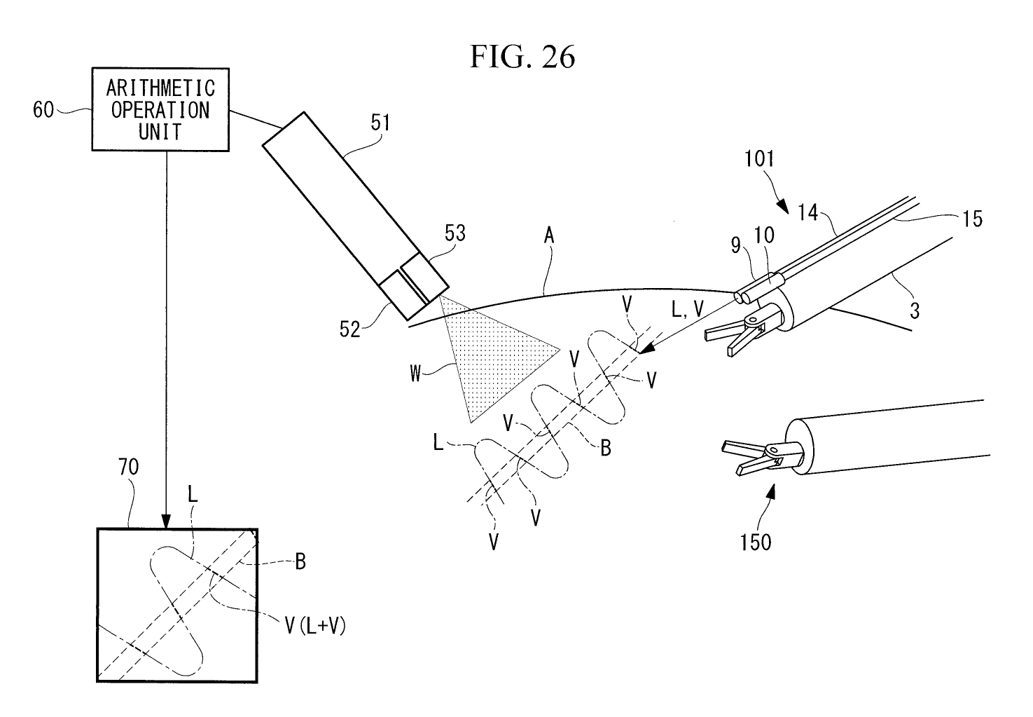

In addition, the blood vessel recognition device 100 of the endoscope apparatus 200 according to this embodiment may include a gripping means 150 for gripping the living body, as shown in FIG. 26.

In this case, blood vessel recognition can be performed independently of a treatment tool 101, without having to additionally mount blood vessel recognition means on the treatment tool 101. In this case, blood vessel recognition can be performed without increasing the outer diameter of the treatment tool 101. In particular, in an energy device for surgical treatment, in which living tissue, fat, and a small-diameter blood vessel from which bleeding can be easily arrested are resected or coagulated and sealed via ultrasound and/or high frequency as described above, there is an advantage in that blood vessel recognition can be performed while avoiding the risk that mist originating from the affected area as a result of the motion of the treatment tool 101 adheres to the blood vessel recognition device 100.

In addition, in this embodiment, items of information about the acquired blood vessel may be joined to one another and may be displayed on the display unit 70 as an independent blood vessel image.

Because the image acquisition device 500 is generally held by a surgery assistant, only a smaller amount of shift in the field of view may occur in this case than in a case in which the image acquisition device 500 is held by the operator himself/herself. If this is the case, the operator may be informed of the blood vessel position by continuously displaying the blood vessel recognition position while omitting the correction of a shift in the field of view.

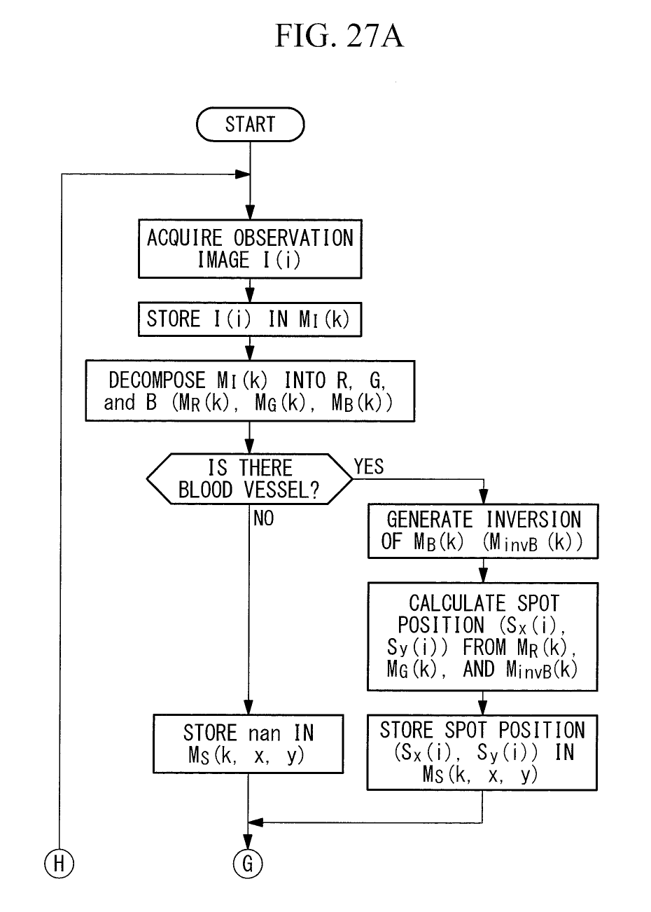

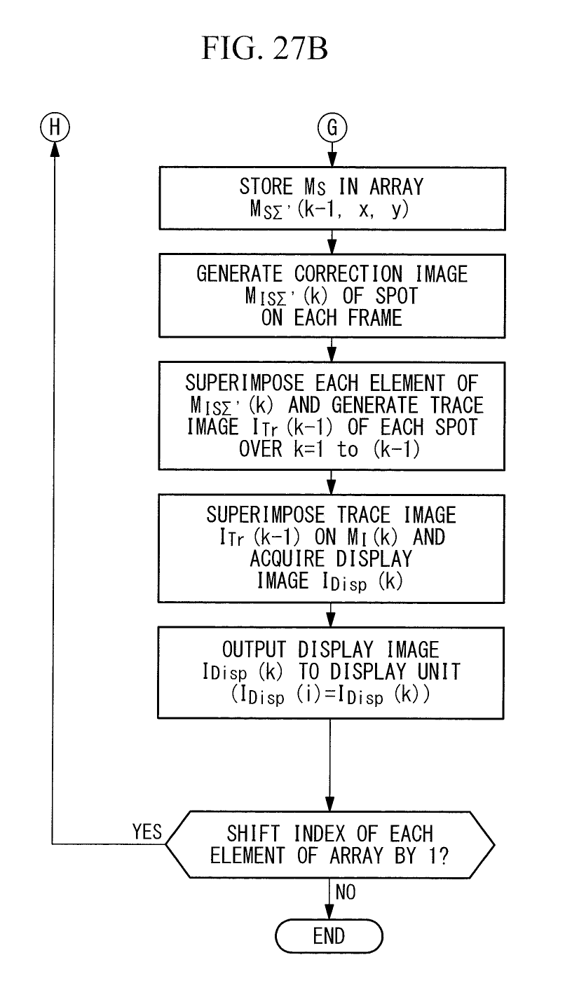

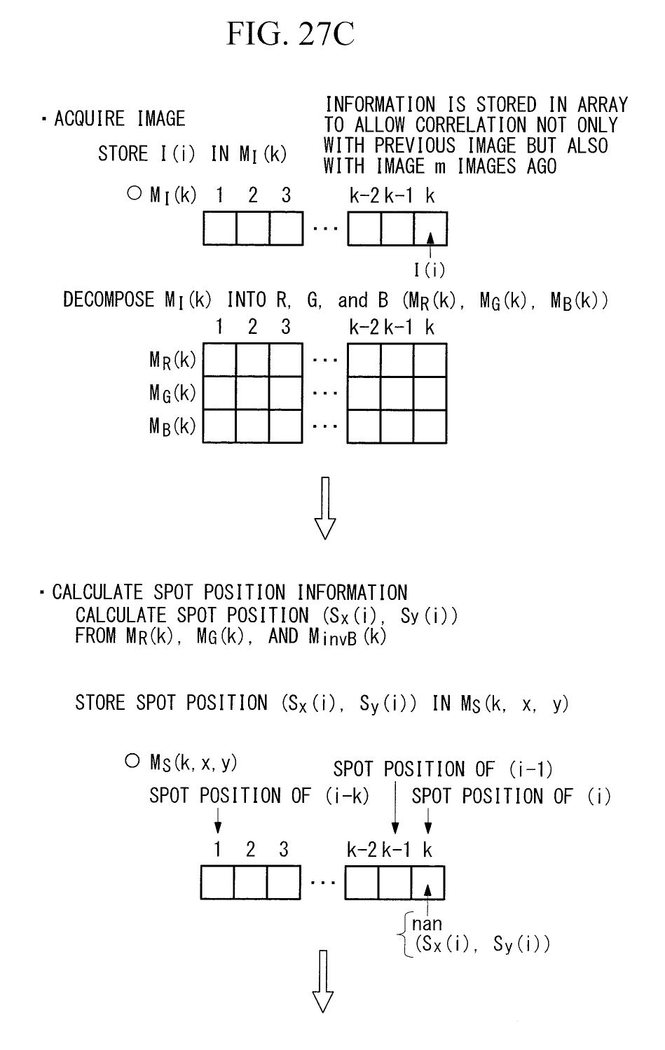

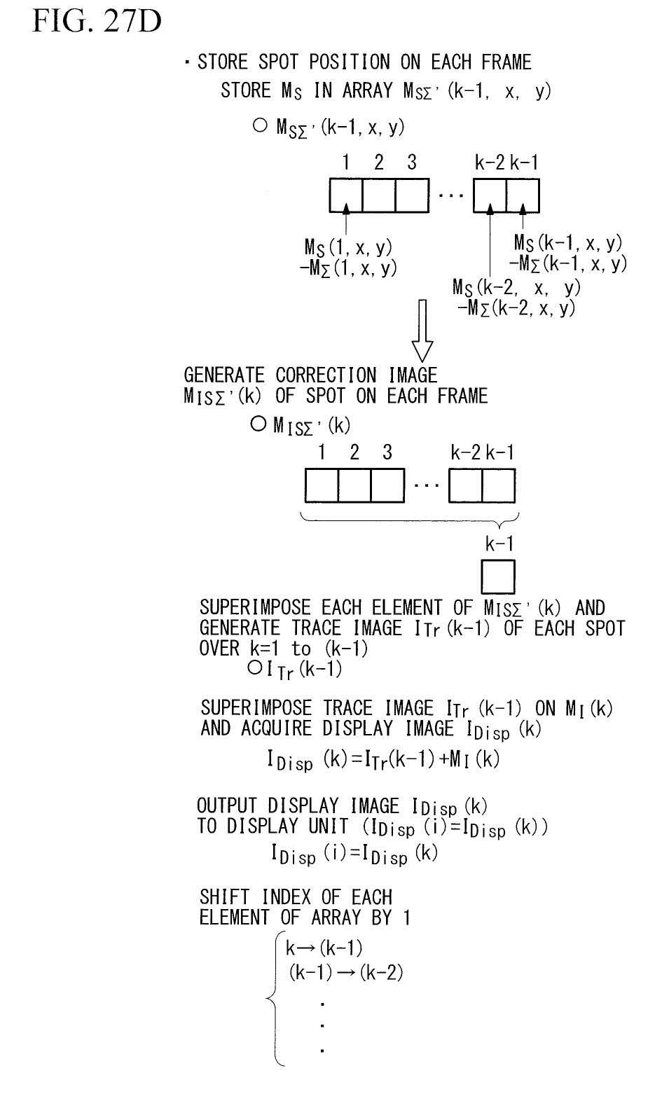

This can be achieved by performing processing and display according to the flow in FIGS. 27A to 27D. In this determination method shown in FIGS. 27A to 27D, the steps in the above-described flow in FIGS. 15A to 15F, i.e., the steps for calculation of displacement via image correlation and position correction, are omitted.

Flow (1) will be described below. First, an observation image I(i) is acquired in the i-th iteration.

This observation image I(i) is stored in the k-th element M_{I}(k) of the array M_{I}.

M_{T}(k) is decomposed into the channels R, G, and B to acquire M_{R}(k), M_{G}(k), and M_{B}(k).

Next, different processing is performed depending on the result of the blood vessel determination. First, if it is determined that there is a blood vessel (YES, i.e., True in the figure), then M_{invB}(k), which is the inversion of M_{B}(k), is generated, and the position at which it is determined that a blood vessel is present, i.e., the spot position (Sₓ(i), S_{y}(i)), is calculated from the three images M_{R}(k), M_{G}(k), and M_{invB}(k). This spot position (Sₓ(i), S_{y}(i)) is substituted into the k-th element M_{S}(k, X, Y) of the array M_{S}. Here, for the sake of convenience, the coordinates (Sₓ(k), S_{y}(k)) of the spot are denoted as M_{S}(k, X, Y), to indicate the k-th element of M_{S}. On the other hand, if it is determined that there are no blood vessels (NO, i.e., false in the figure), then nan, indicating that no corresponding spot position is present, is substituted into the k-th element M_{S}(k, X, Y) of the array M_{S}.

Next, the spot position to be superimposed is stored in the array M_{SΣ′}(k−1, x, y).

A correction image M_{ISΣ′}(k) is produced by placing a mark of a desired size on the image of each frame on the basis of this array information. The size of the mark may be the size in accordance with the actual spot diameter, or an image made to have a hue that is easy to identify is also acceptable.

By superimposing each element of these correction images M_{ISΣ′}(k), a trace image I_{Tr}(k−1) of the marks over k=1 to (k−1) is generated. Here, if k is 2, then a single mark is produced, and if k>2, then the operation of superimposing a plurality of marks is performed, and therefore, a trace image of the spot positions (marks) on the frames is obtained.

Next, by superimposing the trace image I_{Tr}(k−1) on the M_{I}(k), i.e., observation image I(i), a display image I_{Disp}(k) is acquired.

The display image I_{Disp}(k) is output to the display unit 70 (I_{Disp}(i)=I_{Disp}(k) in the case of measurement i).

The index indicating each element of array data is shifted to the side smaller by one at the end of the processing loop. By doing so, information acquired on the previous frame is accumulated.

The next observation image I(i+1) is acquired via the loop, and the same processing is continued.

The length of the accumulated information is specified by setting the size k of the array as appropriate, and hence the time period for which the trace image of blood vessel detection positions is displayed can be set.

It is preferable that an ON/OFF button (operating button) for blood vessel recognition be provided so as to allow the operation of blood vessel recognition to be temporarily interrupted, for cases in which the treatment tool 101 provided with the blood vessel recognition device 100 and/or the endoscope apparatus 200 is moved to another affected area or observation field of view and blood vessel recognition is resumed or for cases in which only the operation for treatment of the affected area with the treatment tool 101 is performed before/after or during the operation of blood vessel recognition for the use of the trace image to which the flow in FIGS. 27A to 27D is applied. By doing so, even if a surgery assistant secures a stable field of view, display of a trace image with low correlation can be prevented in a case where the treatment tool 101 provided with the blood vessel recognition device 100 and/or the endoscope apparatus 200 is moved for purposes other than blood vessel recognition. Particularly in a case where correction of a shift in the field of view is not performed assuming that there is only little shift in the field of view with an image acquisition device 500 as in this embodiment, an advantage is afforded in that recognized blood vessel information can be additionally and promptly displayed as a trace image in response to the user's intention to perform blood vessel recognition in such a manner that the user who operates the treatment tool 101 provided with the blood vessel recognition device 100 stops radiation of a laser beam for blood vessel recognition by turning OFF the ON/OFF button when he/she wishes to operate the treatment tool 101 for gripping purposes or the user manually switches the ON/OFF button to ON only when he/she wishes to perform the operation for blood vessel recognition. When the ON/OFF button is turned ON again in this manner, the above-described flow in FIGS. 27A to 27D can be resumed by causing the endoscope apparatus 200 to detect a laser beam radiation position.

In addition, the trace image to be displayed in the loop does not need to display all information based on the measured image. By doing so, the processing speed can be enhanced, and furthermore, display becomes more visually recognizable by eliminating excessive trace images.

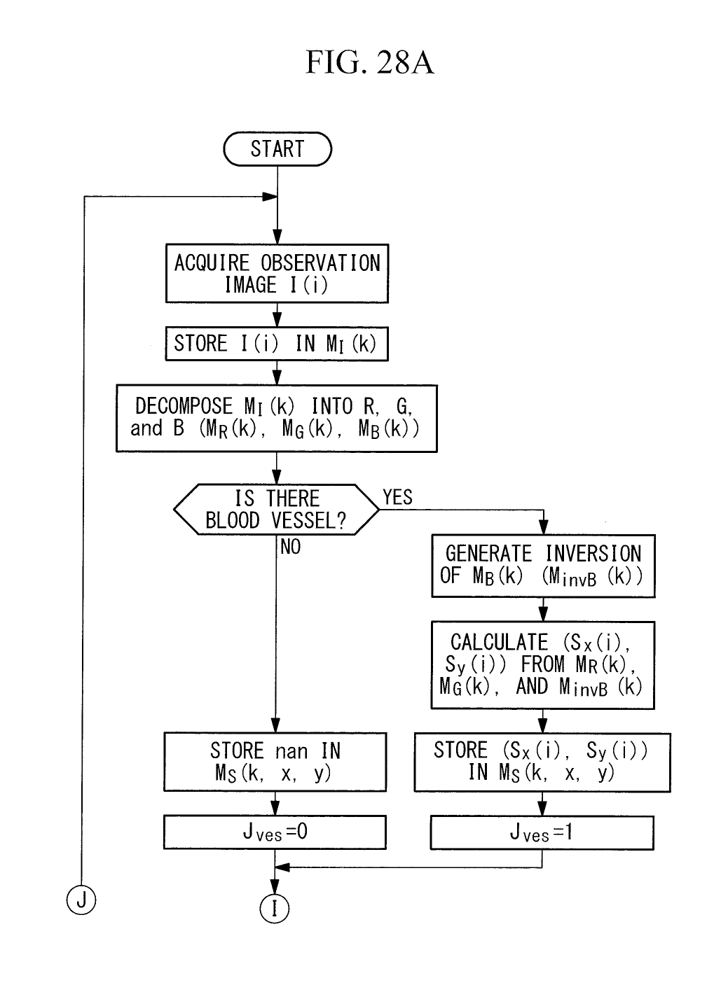

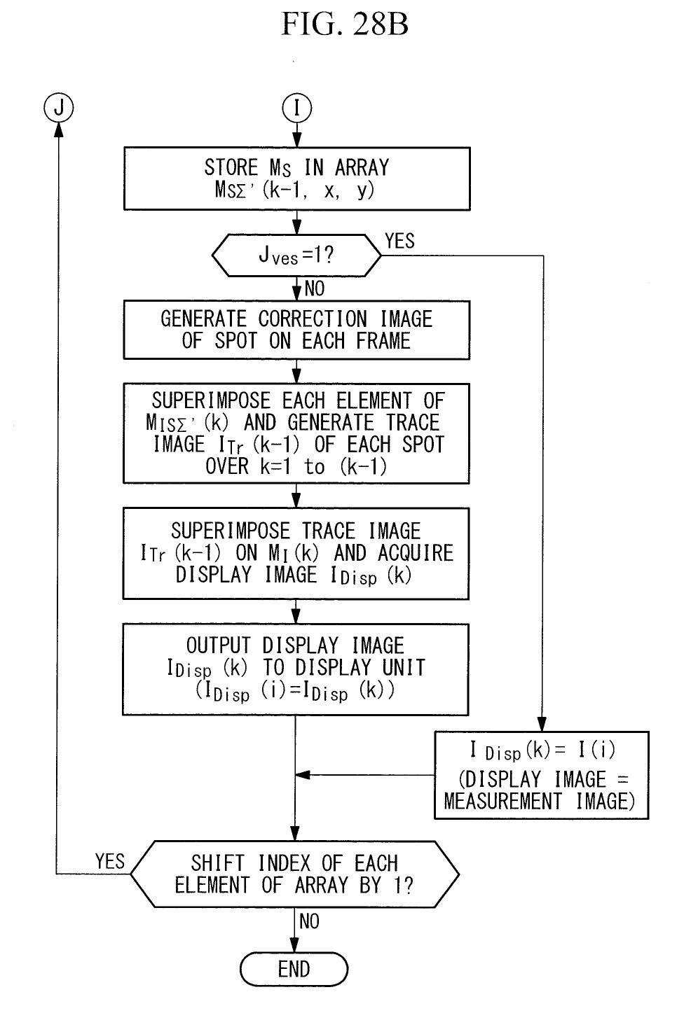

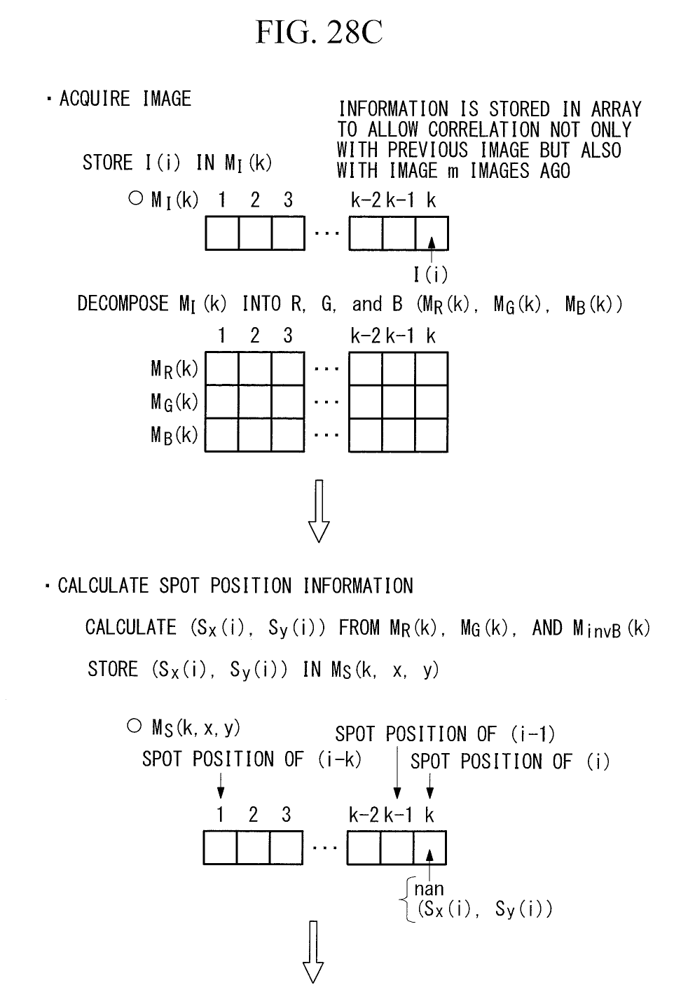

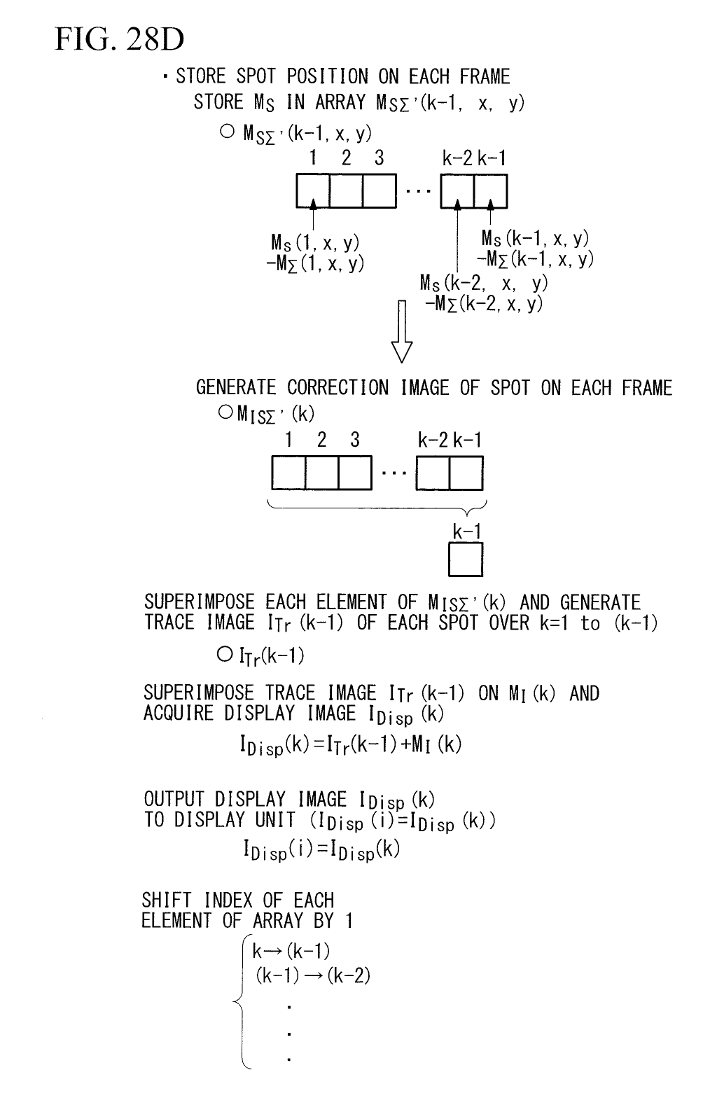

Although flow (2) in FIGS. 28A to 28D is identical to flow (1) in FIGS. 27A to 27D, 1 is substituted into the determination constant Jᵥₑₛ in the case of YES, and 0 is substituted into the determination constant Jᵥₑₛ in the case of NO after blood vessel recognition is performed.

Furthermore, a trace is displayed only when the determination constant Jᵥₑₛ is 0 downstream of the flow. By doing so, it is possible to add a function for prohibiting superimposition of historical records of determination (prohibiting tracing at the positions at which it is determined that there was a blood vessel in the past) while a determination that there is a blood vessel is maintained. Therefore, even if the operator repeatedly scans the same site of living tissue, only newly detected blood vessel information is progressively added while preventing a crowded trace display. Such prohibition of superimposition display can maintain accuracy, as long as the resolution of the blood vessel recognition device 100 is not exceeded. The treatment tool 101 that is provided with the blood vessel recognition device 100 having a function for prohibiting such superimposition display affords an advantage in that it becomes even easier to identify a real-time determination spot because the blood vessel near the affected area can be clearly and comprehensively displayed merely by the operator or the surgery assistant scanning the blood vessel recognition device 100 randomly.

In the case of the complementary color optical system, it is possible to calculate a spot position on the image by performing the same processing as in step 1.1 using information obtained by conventional RGB conversion.

In addition, the above-described embodiments may be provided with an interface for allowing the operator or the surgery assistant to switch among the determination methods illustrated in FIGS. 15A to 15F, FIGS. 16A to 16F, FIGS. 27A to 27D, and FIGS. 28A to 28D using a trace-display-method selection switch (switching unit), which is not shown in the figure. By doing so, a trace display method can be selected on the screen according to the determination of the operator, enabling display according to the surgical situation.

Although a laser beam is used for blood vessel detection in the above-described embodiments, the embodiments are not limited to this. Instead, other coherent light with matched phases may be used.

In addition, although the near-infrared region is used as the wavelength of the laser beam in the above-described embodiments, the embodiments are not limited to this. Instead, NBI (Narrow Band Imaging: Narrow Band Imaging method) may be used.

### REFERENCE SIGNS LIST

1 Treatment tool
2 Control unit
3 Body section
8 Exploration laser light source
9 Light-emitting section
10 Light receiving section
11 Light detection unit
12 Frequency analysis unit
13 Determination unit
16 Visible light source
51 Endoscope body section
52 Illuminating section
53 Image-capturing unit
60 Arithmetic operation unit
70 Display unit
83 Trocar
84 Rotational position sensor (sensor)
85 Insertion length sensor (sensor)
86 Position sensor (sensor)
100 Blood vessel recognition device
101 Treatment tool
150 Gripping means
200 Endoscope apparatus
500 Image acquisition device
A Living tissue
B Blood vessel
C Red blood cell
I Observation image
L Exploration laser beam
S Scattered light
V Visible light
W White light
WI Observation image light
E Abdominal cavity

1. An image processing apparatus comprising:
an image acquisition device for observing a living body;
a blood vessel recognition device for irradiating the living body with an exploration laser beam and recognizing a blood vessel via a laser Doppler method;
an arithmetic operation unit that calculates a first laser spot position on a first frame acquired by the image acquisition device when a blood vessel is recognized by the blood vessel recognition device and that adds a mark corresponding to the first laser spot position to a position on a second frame corresponding to the first frame, the second frame being acquired at a time different from the time of the first frame; and
a display unit for displaying, on an image acquired by the image acquisition device, a plurality of the marks that are added by the arithmetic operation unit as a result of the blood vessel being recognized at a plurality of different times.

2. The image processing apparatus according to claim 1, wherein the arithmetic operation unit further calculates position shift information of the second frame relative to the first laser spot position, calculates a correction position on the second frame on the basis of the calculated position shift information, the correction position corresponding to the first laser spot position, and adds the mark to the correction position on the second frame.

3. The image processing apparatus according to claim 1, wherein, if it is determined by the blood vessel recognition device via the laser Doppler method that the blood vessel is present, the living body is irradiated with an index laser beam, and
the arithmetic operation unit calculates a spot position of the index laser beam.

4. The image processing apparatus according to claim 3, wherein in the blood vessel recognition device, a radiation spot of at least one of the exploration laser beam and the index laser beam has a specific shape different from an Airy pattern.

5. The image processing apparatus according to claim 3, wherein the blood vessel recognition device radiates the index laser beam so that the radiation spot of the index laser beam takes a specific shape different from an Airy pattern, and the arithmetic operation unit calculates the laser spot position using an image correlation with a separately prepared group of reference images of the radiation spot of the index laser beam.

6. The image processing apparatus according to claim 1, wherein the radiation spot of the exploration laser beam has a concentric-ring shape, and
the arithmetic operation unit calculates the laser spot position using an image correlation between the shape of the spot of the exploration laser beam and a separately prepared group of concentric-ring pattern reference images.

7. The image processing apparatus according to claim 2, wherein the arithmetic operation unit acquires the position shift information using an image correlation between the first frame and the second frame that are acquired by the image acquisition device.

8. The image processing apparatus according to claim 7, wherein when performing the image correlation, the arithmetic operation unit acquires a transformation image based on any one or a combination of any of enlargement/reduction, angle, and skew of one of the first frame and the second frame that are different from each other and acquires the image correlation using the transformation image.

9. The image processing apparatus according to claim 2, further comprising:
an insertion section that has the image acquisition device and that is inserted into the body of a patient via a trocar; and
a sensor that acquires a relative position between the insertion section and the trocar,
wherein the arithmetic operation unit calculates the position shift information on the basis of the relative position acquired by the sensor.

10. The image processing apparatus according to claim 9, wherein the sensor is provided on the insertion section.

11. The image processing apparatus according to claim 1, wherein the blood vessel recognition device has a grip section for gripping the living body.

12. The image processing apparatus according to claim 11, wherein the blood vessel recognition device has an operating button that manually switches ON/OFF a blood vessel recognition operation.

13. An endoscope apparatus comprising:
an image acquisition device for observing a living body;
a blood vessel recognition device for irradiating the living body with a laser beam and recognizing a blood vessel via a laser Doppler method;
a switching unit for switching between a method for calculating a laser spot position on a frame acquired by the image acquisition device when a blood vessel is recognized by the blood vessel recognition device, calculating position shift information of the frame relative to a frame that is acquired by the image acquisition device at a time different from the time of the frame, calculating, on the basis of the calculated position shift information, a correction position on the frame acquired at a different time, the correction position being a position corrected for the laser spot position on the frame, and adding a mark corresponding to the correction position to the frame acquired at a different time to display the mark on the frame acquired at a different time and a method for calculating a laser spot position on a frame acquired by the image acquisition device when a blood vessel is recognized by the blood vessel recognition device and adding a mark corresponding to the laser spot position to the frame acquired at a different time to display the mark on the frame acquired at a different time;
an arithmetic operation unit for adding the mark selected by the switching unit to the frame acquired at a different time and displaying the mark; and,
a display unit for displaying the mark added by the arithmetic operation unit on an image acquired by the image acquisition device.
